(19) United States
(12) Patent Application Publication
(10) Pub. No.: US 2024/0352484 A1
(43) Pub. Date: Oct. 24, 2024

(54) REWARD SYSTEM ACTIVATION FOR THERAPEUTIC PURPOSES

(71) Applicants: Technion Research & Development Foundation Limited, Haifa (IL); Rambam Med-Tech Ltd., Haifa (IL)

(72) Inventors: Asya ROLLS, Haifa (IL); Lior GEPSTEIN, Haifa (IL); Hedva HAYKIN, Haifa (IL); Hilla AZULAY-DEBBY, Haifa (IL)

(73) Assignees: Technion Research & Development Foundation Limited, Haifa (IL); Rambam Med-Tech Ltd., Haifa (IL)

(21) Appl. No.: 18/638,775

(22) Filed: Apr. 18, 2024

Related U.S. Application Data
(60) Provisional application No. 63/460,054, filed on Apr. 18, 2023.

(51) Int. Cl. C12N 15/86; A61B 5/055

(52) U.S. Cl. CPC C12N 15/86; A61B 5/055; C12N 2750/14143

(57) ABSTRACT

Provided is reward system activation for therapeutic purposes. Accordingly there is provided a method of treating a disease in a subject in need thereof, the method comprising subjecting the subject to a treatment module which activates the reward system of the subject.

### RELATED APPLICATION(S)

This application claims the benefit of priority under 35 USC § 119 (e) of U.S. Provisional Patent Application No. 63/460,054 filed on Apr. 18, 2023, the contents of which are all incorporated by reference as if fully set forth herein in their entirety.

### FIELD AND BACKGROUND OF THE INVENTION

The present invention, in some embodiments thereof, relates to reward system activation for therapeutic purposes.

The brain's reward system, specifically the dopaminergic neurons in the ventral tegmental area (VTA), constitutes a key neuronal network whose activation mediates positive emotions, expectations, and motivation [Hyman S E, et al. Annu Rev Neurosci. (2006) 29:565-98; Tsai H C et al, Science. (2009) 324(5930):1080-4]. The VTA receives inputs from the lateral dorsal tegmentum (LDTg), lateral habenula (LHb), lateral hypothalamus (LH) and from the amygdala (Amy). It projects to the nucleus accumbens (NAc), the medial prefrontal cortex (mPFC) and the amygdala. Additional direct and indirect pathways also exist, connecting the various structures comprising the reward system. The dopaminergic projections from the VTA to components of the limbic system are causally associated with motivated behavior and reward perception [e.g. Schultz W. J Neurophysiol (1998) 80(1):1-27].

Previous studies indicated a causal relationship between the activity of the reward system and the immune response and modulating activity of dopaminergic neurons of the VTA have been suggested as a treatment modality for several diseases including e.g. cancer, infectious diseases, immune-deficient or autoimmune diseases [Ben-Shaanan T L et al. Nat Med. (2016) 22(8):940-4; Ben-Shaanan T L et al. Nat Commun (2018) 9(1):2723; and International Patent Application Publication Nos: WO2015079439 and WO2020178820].

### SUMMARY OF THE INVENTION

According to an aspect of some embodiments of the present invention there is provided a method of treating a disease selected from the group consisting of liver disease, kidney disease, psoriatic arthritis, systemic lupus erythematosus (SLE), rheumatoid arthritis, anti-neutrophil cytoplasmic antibody-associated vasculitis, synaptic change in multiple sclerosis, amyotrophic lateral sclerosis (ALS), ALS9, ALS1, neonatal Myasthenia Gravis, Crohn's disease, Huntington disease, oculopharyngeal muscular dystrophy, ataxia-spinocerebellar ataxia 7, spinocerebellar ataxia 1, autosomal dominant cerebellar ataxia, hemolytic anemia, septic shock, stroke, Timothy Syndrome, Long Qt Syndrome, schizophrenia, bipolar disorder, major depression, anxiety, autism, ADHD, sepsis, viral lower respiratory tract infections, chronic heart failure, Diabetes type 2, hemodialysis, dilated cardiomyopathy, lipid storage myopathy, peanut allergy, pericarditis, rheumatic disease, Camptodactyly-Arthropathy-Coxa Vara-Pericarditis Syndrome, amyloidosis, Alzheimer's disease, Parkinson's disease, Laryngeal disease, laryngitis, laryngomalacia, spherocytosis, type 5, hereditary spherocytosis, Bardet-Biedl Syndrome, schizophrenia, hyperhomocysteinemia, neural tube defects, homocystinuria, placental abruption, coronary artery disease, xerophthalmia, Noonan syndrome 6, succinic semialdehyde dehydrogenase deficiency, thrombophilia due to thrombin defect, lecithin:cholesterol acyltransferase deficiency, fish-eye disease, Tangier Disease, hypoalphalipoproteinemia, phosphoserine phosphatase deficiency, arthrogryposis, distal arthrogryposis, distal arthrogryposis type 1C, multidrug-resistant tuberculosis, Meester-Locys syndrome, Monckeberg arteriosclerosis, familial Mediterranean fever, mitochondrial complex Ii deficiency, mitochondrial complex Ii deficiency nuclear type 2, amyotrophic neuralgia, brachial plexus neuropathy, thrombosis and nephrotic syndrome type 5, Agenesis of corpus callosum, cardiac, ocular, and genital syndrome (ACOGS), hematuria, myopathy, lactic acidosis, sideroblastic anemia, snail allergy, crustacean allergy, carnitine-acylcarnitine translocase deficiency, Retinitis pigmentosa, Retinitis pigmentosa 24, pulmonary fibrosis, idiopathic and nasopharyngitis chondrodysplasia punctata 2 X-Linked dominant, Mend syndrome, bare lymphocyte syndrome, bare lymphocyte syndrome type Ii, nominal aphasia, gastrointestinal anthrax, barbiturate dependence, intellectual disability syndrome with long QT, myoclonus, myoclonus familial 1, alcoholic pancreatitis, polymicrogyria, perisylvian, with cerebellar hypoplasia and arthrogryposis (NEDSPLB), arthrogryposis, heparin cofactor Ii deficiency, pyridoxamine 5-prime-phosphate oxidase deficiency, acid-labile subunit deficiency, neurodevelopmental disorder with microcephaly hypotonia and variable brain anomalies (NMIHBA), ulnar neuropathy, developmental delay, brain abnormalities including ventriculomegaly and brain atrophy, optic nerve abnormalities, Diamond-Blackfan anemia, Diamond-Blackfan anemia 13, X-linked myopathy with postural muscle atrophy, Emery-Dreifuss muscular dystrophy 5, Smith-Magenis syndrome, spinocerebellar ataxia autosomal recessive 24, developmental and epileptic encephalopathy 44, Charcot-Marie tooth disease axonal type 2P, autosomal dominant mental retardation 20, 3-methylcrotonyl-coa carboxylase deficiency, St. Louis encephalitis, eczema herpeticum, porphyria-acute Hepatic cutanea tarda variegate, tyrosinemia type I, Hermansky-Pudlak syndrome, hyperbiliverdinemia, cholestasis, pneumothorax, mild cognitive impairment, multiple mitochondrial dysfunctions syndrome 2 with hyperglycinemia, familial isolated dilated cardiomyopathy, caspase 8 deficiency, villonodular synovitis, hantavirus pulmonary syndrome, myotonic dystrophy 1 and 2, palmoplantar keratoderma bothnian type, Silver-Russell syndrome 1, xeroderma pigmentosum complementation group E, persistent hyperplastic primary vitreous (PHPV), trichostrongylosis, osteogenesis imperfecta, dentinogenesis imperfecta, osteopetrosis, narcolepsy, otopalatodigital syndrome, progressive myoclonus epilepsy, thyroid Crisis, granulomatous disease, lymphadenitis, skeletal tuberculosis, lymphopenia, nonparalytic poliomyelitis, pulmonary hypertension, Acute febrile neutrophilic dermatosis, glaucomatocyclitic crisis, cone-rod dystrophy 2, fundus dystrophy, esophageal diverticulosis, malnutrition and cachexia, wherein the disease is not cancer, in a subject in need thereof, the method comprising subjecting the subject to a treatment module which activates the reward system of the subject, thereby treating the disease in the subject.

According to an aspect of some embodiments of the present invention there is provided a treatment module for activating the reward system of a subject for use in treating a disease selected from the group consisting of liver disease, kidney disease, psoriatic arthritis, systemic lupus erythematosus (SLE), rheumatoid arthritis, anti-neutrophil cytoplasmic antibody-associated vasculitis, synaptic change in multiple sclerosis, amyotrophic lateral sclerosis (ALS), ALS9, ALS1, neonatal Myasthenia Gravis, Crohn's disease, Huntington disease, oculopharyngeal muscular dystrophy, ataxia-spinocerebellar ataxia 7, spinocerebellar ataxia 1, autosomal dominant cerebellar ataxia, hemolytic anemia, septic shock, stroke, Timothy Syndrome, Long Qt Syndrome, schizophrenia, bipolar disorder, major depression, anxiety, autism, ADHD, sepsis, viral lower respiratory tract infections, chronic heart failure, Diabetes type 2, hemodialysis, dilated cardiomyopathy, lipid storage myopathy, peanut allergy, pericarditis, rheumatic disease, Camptodactyly-Arthropathy-Coxa Vara-Pericarditis Syndrome, amyloidosis, Alzheimer's disease, Parkinson's disease, Laryngeal disease, laryngitis, laryngomalacia, spherocytosis, type 5, hereditary spherocytosis, Bardet-Biedl Syndrome, schizophrenia, hyperhomocysteinemia, neural tube defects, homocystinuria, placental abruption, coronary artery disease, xerophthalmia, Noonan syndrome 6, succinic semialdehyde dehydrogenase deficiency, thrombophilia due to thrombin defect, lecithin: cholesterol acyltransferase deficiency, fish-eye disease, Tangier Disease, hypoalphalipoproteinemia, phosphoserine phosphatase deficiency, arthrogryposis, distal arthrogryposis, distal arthrogryposis type 1C, multidrug-resistant tuberculosis, Meester-Loeys syndrome, Monckeberg arteriosclerosis, familial Mediterranean fever, mitochondrial complex Ii deficiency, mitochondrial complex Ii deficiency nuclear type 2, amyotrophic neuralgia, brachial plexus neuropathy, thrombosis and nephrotic syndrome type 5, Agenesis of corpus callosum, cardiac, ocular, and genital syndrome (ACOGS), hematuria, myopathy, lactic acidosis, sideroblastic anemia, snail allergy, crustacean allergy, carnitine-acylcarnitine translocase deficiency, Retinitis pigmentosa, Retinitis pigmentosa 24, pulmonary fibrosis, idiopathic and nasopharyngitis chondrodysplasia punctata 2 X-Linked dominant, Mend syndrome, bare lymphocyte syndrome, bare lymphocyte syndrome type Ii, nominal aphasia, gastrointestinal anthrax, barbiturate dependence, intellectual disability syndrome with long QT, myoclonus, myoclonus familial 1, alcoholic pancreatitis, polymicrogyria, perisylvian, with cerebellar hypoplasia and arthrogryposis (NEDSPLB), arthrogryposis, heparin cofactor Ii deficiency, pyridoxamine 5-prime-phosphate oxidase deficiency, acid-labile subunit deficiency, neurodevelopmental disorder with microcephaly hypotonia and variable brain anomalies (NMIHBA), ulnar neuropathy, developmental delay, brain abnormalities including ventriculomegaly and brain atrophy, optic nerve abnormalities, Diamond-Blackfan anemia, Diamond-Blackfan anemia 13, X-linked myopathy with postural muscle atrophy, Emery-Dreifuss muscular dystrophy 5, Smith-Magenis syndrome, spinocerebellar ataxia autosomal recessive 24, developmental and epileptic encephalopathy 44, Charcot-Marie tooth disease axonal type 2P, autosomal dominant mental retardation 20, 3-methylcrotonyl-coa carboxylase deficiency, St. Louis encephalitis, eczema herpeticum, porphyria-acute Hepatic cutanea tarda variegate, tyrosinemia type I, Hermansky-Pudlak syndrome, hyperbiliverdinemia, cholestasis, pneumothorax, mild cognitive impairment, multiple mitochondrial dysfunctions syndrome 2 with hyperglycinemia, familial isolated dilated cardiomyopathy, caspase 8 deficiency, villonodular synovitis, hantavirus pulmonary syndrome, myotonic dystrophy 1 and 2, palmoplantar keratoderma bothnian type, Silver-Russell syndrome 1, xeroderma pigmentosum complementation group E, persistent hyperplastic primary vitreous (PHPV), trichostrongylosis, osteogenesis imperfecta, dentinogenesis imperfecta, ostcopetrosis, narcolepsy, otopalatodigital syndrome, progressive myoclonus epilepsy, thyroid Crisis, granulomatous disease, lymphadenitis, skeletal tuberculosis, lymphopenia, nonparalytic poliomyelitis, pulmonary hypertension, Acute febrile neutrophilic dermatosis, glaucomatocyclitic crisis, cone-rod dystrophy 2, fundus dystrophy, esophageal diverticulosis, malnutrition and cachexia, wherein the disease is not cancer.

According to some embodiments of the invention, the liver disease is selected from the group consisting of hepatitis A, hepatitis B and hepatitis C.

According to some embodiments of the invention, the kidney disease is selected from the group consisting of C3 glomerulopathy, chronic kidney disease, acute glomerulonephritis, membranoproliferative glomerulonephritis, atypical hemolytic uremic syndrome, Lupus nephritis, podocytopathy, diabetic nephropathy, albuminuria, autosomal dominant polycystic kidney disease, ischemia/reperfusion-induced acute kidney injury, chronic renal failure and progressive proteinuric nephropathy.

According to an aspect of some embodiments of the present invention there is provided a method of treating cancer in a subject in need thereof, wherein the cancer is selected from the group consisting of sporadic breast cancer, triple-negative breast cancer-luminal-like subtype wild-type p53, hepatocellular carcinoma, larynx cancer papillary, nasopharyngeal carcinoma, cystadenocarcinoma, non-medullary thyroid cancer, myxosarcoma, renal cell carcinoma, nonpapillary renal cell carcinoma, clear cell papillary renal cell carcinoma, skin squamous cell carcinoma, Gliosarcoma, glioblastoma, giant cell glioblastoma, uveal melanoma, chondroblastoma, pancreatic acinar cell adenocarcinoma, petroclival meningioma, granular cell tumor, follicular dendritic cell sarcoma, liver sarcoma, histiocytic sarcoma, epithelioid sarcoma and periapical granuloma, the method comprising subjecting the subject to a treatment module which activates the reward system of the subject, thereby treating the cancer in the subject.

According to an aspect of some embodiments of the present invention there is provided a treatment module for activating the reward system of a subject for use in treating cancer selected from the group consisting of sporadic breast cancer, triple-negative breast cancer-luminal-like subtype wild-type p53, hepatocellular carcinoma, larynx cancer papillary, nasopharyngeal carcinoma, cystadenocarcinoma, non-medullary thyroid cancer, myxosarcoma, renal cell carcinoma, nonpapillary renal cell carcinoma, clear cell papillary renal cell carcinoma, skin squamous cell carcinoma, Gliosarcoma, glioblastoma, giant cell glioblastoma, uveal melanoma, chondroblastoma, pancreatic acinar cell adenocarcinoma, petroclival meningioma, granular cell tumor, follicular dendritic cell sarcoma, liver sarcoma, histiocytic sarcoma, epithelioid sarcoma and periapical granuloma.

According to some embodiments of the invention, the treatment module activates a dopaminergic neuron in the ventral tegmental area (VTA) of the subject or a post synaptic neuron thereof.

According to some embodiments of the invention, the treatment module comprises sensory, auditory, visual and/or chemical stimulation.

According to some embodiments of the invention, the treatment module comprises digital experiences and/or virtual reality stimulation.

According to some embodiments of the invention, the treatment module comprises magnetic stimulation, electric stimulation and/or an ultrasound stimulation.

According to some embodiments of the invention, the magnetic stimulation comprises Transcranial magnetic stimulation (TMS).

According to some embodiments of the invention, the treatment module comprises transfecting a neuron in the reward system with a receptor activated solely by a synthetic ligand (RASSL) and/or designer receptor exclusively activated by designer drugs (DREADD).

According to some embodiments of the invention, the treatment module comprises neurofeedback.

According to some embodiments of the invention, the neurofeedback comprises electroencephalography, functional magnetic resonance imaging, functional near-infrared spectrometry, diffusion-weighted magnetic resonance imaging and/or functional magnetic resonance spectrometry.

According to some embodiments of the invention, the treatment module comprises administering to the subject a dopamine agonist capable of crossing the blood brain barrier.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-H demonstrate the effects of VTA activation on left-ventricular function following myocardial infarction (MI). FIG. 1A shows the Experimental design: Stereotactic viral injection of DREADDs viral vector. Following recovery from the viral injection, LAD ligation is performed, followed by 15 days of daily CNO injections. During this period, cardiac function is assessed and immune analysis is performed. FIG. 1B demonstrates viral expression in the right VTA, manifested by expression of the mCherry reporter. FIG. 1C shows cells expressing the mCherry reporter (red), tyrosine hydroxylase (TH, the limiting enzyme in dopamine production, green) and c-Fos (an early gene indicating activation, purple). FIG. 1D shows long-axis fCMR images of control mice following LAD ligation, showing a reduction in LVEF on day 1, followed by a second reduction in LVEF on day 15; as compared VTA-activated mice showing a reduction in LVEF on day 1, followed by an increased LVEF on day 15. FIG. 1E is a graph demonstrating LVEF on post-operative days 1 and 15 in control and VTA-activated mice. Interaction between control and VTA-activated mice over days; Two-way ANOVA mixed model F(1,22)=25.6, P≤0.0001****. Specific comparisons were found non-significant for VTA-activated mice [t(22)=1.074, p=0.2945] with a significant effect for control mice [t(22)=5.68 p=<0.0001****]. FIG. 1F is a graph demonstrating the difference in LVEF between post-operative days 1 and 15; t(22)=4.753, P>0.0001****. FIG. 1G is a graph demonstrating changes in stroke volume (μl) as measured by fCMR on post-operative days 1 and 15 in control and VTA-activated mice. Interaction between control and VTA-activated mice over days; two-way ANOVA mixed model F(1,22)=31.41, P=<0.0001****. Specific comparisons were found non-significant for control mice [t(22)=1.865 p=0.0755], with a significant effect for VTA-activated mice [t(22)=4.704, p=0.0001***]. FIG. 1H is a graph demonstrating changes in end-diastolic volume (EDV), as measured by fCMR, on post-operative days 1 and 15 in control and VTA-activated mice. Interaction between control and VTA-activated mice over days; two-way ANOVA mixed model F(1,22)=2.878, P=0.1039. Specific comparisons were found significant for control mice [t(22)=2.919 p=0.0079**], and for VTA-activated mice [t(22)=2.777, p=0.011*].

FIGS. 2A-E demonstrate the effects of VTA activation on scar-tissue formation and vascularization 15 days following MI. FIG. 2A shows images of Masson trichrome staining of hearts of control and VTA-activated mice, 15 days following LAD ligation. Each histologic image belongs to a different mice, wherein preparations were taken from the same distance relative to the apex. Collagen fibers are stained in blue. FIG. 2B is a scatter-plot representation of the difference in the proportion of collagen-rich area on day 15 between VTA-activated and control mice; t(14)=2.456 p=0.027*. FIG. 2C shows representative images of control and VTA-activated hearts, 15 days following MI. The slices were stained with actin smooth muscle protein (α-SMA, red) and von Willebrand factor (vWF, green) which is expressed on all endothelial cells. FIG. 2D shows quantification of lumens (α-SMA⁺vWF⁺) by number and the area occupied by those markers. FIG. 2E is a graph demonstrating the diameter distribution of lumens. VTA-activated mice exhibit a bigger fraction of small diameter blood vessels (<50 μm).

FIGS. 3A-D demonstrate full proteomic analysis (LC/MSMS) of cardiac apex samples 4 days post MI. FIG. 3A shows Gene Ontology (GO) analysis, classifying the proteins into several biological processes in which they are involved. The significant difference between VTA-activated and control mice were mostly associated with increase in the amount of immune system processes, including complement activation, negative feedback on cytokine production, organ development, and regulation of triglyceride biosynthesis. VTA-activated mice showed a decrease in a number of processes, including RNA splicing, endocytosis, catabolism, and metabolic processing of nucleotide-sugars. FIG. 3B is a heat map representing the changes in the proteins levels (p<0.15) by hierarchical clustering. FIG. 3C is a Volcano plot presenting the fold change in proteins levels between VTA activated and control mice. Statistically significant proteins (p-value<0.05, FDR<0.0001) are marked with positive difference in red and negative in blue. FIG. 3D shows a GOrilla pathway enrichments analysis highlighting that VTA activation results in alterations in many processes that are related to the recovery process following MI. For example, biological processes resulting in complement activation through the alternative pathway and actin nucleation, the first step in actin polymerization, as well as synthesis of lipoproteins and ATP.

FIGS. 4A-J demonstrate trans-synaptic neuronal tracing of pathways descending from the VTA following MI. FIG. 4A shows the increase in C3 levels in the heart in the VTA-activated group. FIG. 4B shows that the increase in C3 is from the liver. Shown are mRNA levels. FIGS. 4C-H show PRV injections. Specifically, PRV-152 was injected to the liver hilum in C57BL/6J WT mice. Six days later, colons and brains were harvested of for further IF analysis. FIG. 4D-F show representative micrographs of the liver (FIG. 4D), celiac ganglion (FIG. 4E) and spinal cord (FIG. 4F) demonstrating neurons expressing PRV (green). Scale bar: 100 or 1000 μm. FIG. 4G shows a representative composite micrograph of the brain demonstrating PRV+ neurons in the VTA 6 days following PRV injection to the liver hilum. Dopamine producing cells are distinguished by IF staining of tyrosine hydroxylase (TH), the limiting enzyme in dopamine production. Scale bar: 50 μm. FIG. 4H shows quantification of PRV cells in brainstem autonomic nuclei. FIG. 4I shows the change in the scar tissue following VTA activation shows quantification of PRV+ cells in the VTA, and specifically TH+ cells. FIG. 4J shows noradrenaline (NA) levels measured by ELISA in mice's liver 4 days post AMI.

### DESCRIPTION OF SPECIFIC EMBODIMENTS OF THE INVENTION

The present invention, in some embodiments thereof, relates to reward system activation for therapeutic purposes.

Whilst reducing the present invention to practice, the present inventors have uncovered that VTA activation effects an array of pathways intimately involved in the recovery process following myocardial infarction (MI) (see Examples 1-3 of the Examples section which follows). Moreover, proteomics analysis indicated that VTA activation results in alteration of expression of proteins involved in several other diseases—indicating VTA activation as a therapeutic modality for these diseases.

Thus, according to a first aspect of the present invention, there is provided a method of treating a disease selected from the group consisting of liver disease, kidney disease, psoriatic arthritis, systemic lupus erythematosus (SLE), rheumatoid arthritis, anti-neutrophil cytoplasmic antibody-associated vasculitis, synaptic change in multiple sclerosis, amyotrophic lateral sclerosis (ALS), ALS9, ALS1, neonatal Myasthenia Gravis, Crohn's disease, Huntington disease, oculopharyngeal muscular dystrophy, ataxia-spinocerebellar ataxia 7, spinocerebellar ataxia 1, autosomal dominant cerebellar ataxia, hemolytic anemia, septic shock, stroke, Timothy Syndrome, Long Qt Syndrome, schizophrenia, bipolar disorder, major depression, anxiety, autism, ADHD, sepsis, viral lower respiratory tract infections, chronic heart failure, Diabetes type 2, hemodialysis, dilated cardiomyopathy, lipid storage myopathy, peanut allergy, pericarditis, rheumatic disease, Camptodactyly-Arthropathy-Coxa Vara-Pericarditis Syndrome, amyloidosis, Alzheimer's disease, Parkinson's disease, Laryngeal disease, laryngitis, laryngomalacia, spherocytosis, type 5, hereditary spherocytosis, Bardet-Biedl Syndrome, schizophrenia, hyperhomocysteinemia, neural tube defects, homocystinuria, placental abruption, coronary artery disease, xerophthalmia, Noonan syndrome 6, succinic semialdehyde dehydrogenase deficiency, thrombophilia due to thrombin defect, lecithin: cholesterol acyltransferase deficiency, fish-eye disease, Tangier Disease, hypoalphalipoproteinemia, phosphoserine phosphatase deficiency, arthrogryposis, distal arthrogryposis, distal arthrogryposis type 1C, multidrug-resistant tuberculosis, Meester-Locys syndrome, Monckeberg arteriosclerosis, familial Mediterranean fever, mitochondrial complex Ii deficiency, mitochondrial complex Ii deficiency nuclear type 2, amyotrophic neuralgia, brachial plexus neuropathy, thrombosis and nephrotic syndrome type 5, Agenesis of corpus callosum, cardiac, ocular, and genital syndrome (ACOGS), hematuria, myopathy, lactic acidosis, sideroblastic anemia, snail allergy, crustacean allergy, carnitine-acylcarnitine translocase deficiency, Retinitis pigmentosa, Retinitis pigmentosa 24, pulmonary fibrosis, idiopathic and nasopharyngitis chondrodysplasia punctata 2 X-Linked dominant, Mend syndrome, bare lymphocyte syndrome, bare lymphocyte syndrome type Ii, nominal aphasia, gastrointestinal anthrax, barbiturate dependence, intellectual disability syndrome with long QT, myoclonus, myoclonus familial 1, alcoholic pancreatitis, polymicrogyria, perisylvian, with cerebellar hypoplasia and arthrogryposis (NEDSPLB), arthrogryposis, heparin cofactor Ii deficiency, pyridoxamine 5-prime-phosphate oxidase deficiency, acid-labile subunit deficiency, neurodevelopmental disorder with microcephaly hypotonia and variable brain anomalies (NMIHBA), ulnar neuropathy, developmental delay, brain abnormalities including ventriculomegaly and brain atrophy, optic nerve abnormalities, Diamond-Blackfan anemia, Diamond-Blackfan anemia 13, X-linked myopathy with postural muscle atrophy, Emery-Dreifuss muscular dystrophy 5, Smith-Magenis syndrome, spinocerebellar ataxia autosomal recessive 24, developmental and epileptic encephalopathy 44, Charcot-Marie tooth disease axonal type 2P, autosomal dominant mental retardation 20, 3-methylcrotonyl-coa carboxylase deficiency, St. Louis encephalitis, eczema herpeticum, porphyria-acute Hepatic cutanea tarda variegate, tyrosinemia type I, Hermansky-Pudlak syndrome, hyperbiliverdinemia, cholestasis, pneumothorax, mild cognitive impairment, multiple mitochondrial dysfunctions syndrome 2 with hyperglycinemia, familial isolated dilated cardiomyopathy, caspase 8 deficiency, villonodular synovitis, hantavirus pulmonary syndrome, myotonic dystrophy 1 and 2, palmoplantar keratoderma bothnian type, Silver-Russell syndrome 1, xeroderma pigmentosum complementation group E, persistent hyperplastic primary vitreous (PHPV), trichostrongylosis, osteogenesis imperfecta, dentinogenesis imperfecta, osteopetrosis, narcolepsy, otopalatodigital syndrome, progressive myoclonus epilepsy, thyroid Crisis, granulomatous disease, lymphadenitis, skeletal tuberculosis, lymphopenia, nonparalytic poliomyelitis, pulmonary hypertension, Acute febrile neutrophilic dermatosis, glaucomatocyclitic crisis, cone-rod dystrophy 2, fundus dystrophy, esophageal diverticulosis, malnutrition and cachexia, wherein said disease is not cancer, in a subject in need thereof, the method comprising subjecting the subject to a treatment module which activates the reward system of the subject, thereby treating the disease in the subject.

According to an additional or an alternative aspect of the present invention, there is provided a method of treating cancer in a subject in need thereof, wherein said cancer is selected from the group consisting of sporadic breast cancer, triple-negative breast cancer-luminal-like subtype wild-type p53, hepatocellular carcinoma, larynx cancer papillary, nasopharyngeal carcinoma, cystadenocarcinoma, non-medullary thyroid cancer, myxosarcoma, renal cell carcinoma, nonpapillary renal cell carcinoma, clear cell papillary renal cell carcinoma, skin squamous cell carcinoma, Gliosarcoma, glioblastoma, giant cell glioblastoma, uveal melanoma, chondroblastoma, pancreatic acinar cell adenocarcinoma, petroclival meningioma, granular cell tumor, follicular dendritic cell sarcoma, liver sarcoma, histiocytic sarcoma, epithelioid sarcoma and periapical granuloma, the method comprising subjecting the subject to a treatment module which activates the reward system of the subject, thereby treating the cancer in the subject.

Hence, the methods disclosed herein comprise subjecting the subject to a treatment module which activated the reward system of the subject.

As used herein the term “reward system” refers to a collection of brain structures and neuronal pathways that are responsible for reward-related cognition, including, but not limited to, associative learning, incentive salience (i.e., motivation and “wanting”, desire, or craving for a reward), and positively-valenced emotions, particularly emotions that involve pleasure (i.e., hedonic “liking”). The reward circuitry is comprised of striatal, limbic and pre-frontal cortical structures, including the ventral tegmental area (VTA), NAc, hippocampus and amygdala and postsynaptic neurons thereof and comprise cell many types including glutamatergic interneurons, GABAergic medium spiny neurons (MSNs), and dopaminergic projection neurons.

As used herein, the term “ventral tegmental area (VTA)”, also known as the ventral tegmental area of Tsai, refers to a group of neurons located close to the midline on the floor of the midbrain. The VTA comprises mainly dopaminergic neurons and GABAergic neurons. Typically, the dopaminergic neurons are excited during the “expectation to reward” and the reward itself. The gap between them is calculated and defined as reward prediction errors, driving learning processes. The GABAergic neurons on the other hand inhibit the activity of the dopaminergic neurons and therefore excited from the beginning of the expectation of the reward and during punishment. The activity of the dopaminergic neurons is the most associated one with the VTA and the reward system. The VTA receives inputs from the lateral dorsal tegmentum (LDTg), lateral habenula (LHb), lateral hypothalamus (LH) and from the amygdala (Amy); and projects to the nucleus accumbens (NAc), the medial prefrontal cortex (mPFC), the amygdala, hippocampus, cingulate gyrus and even olfactory bulb.

As used herein, “activating the reward system” refers to inducing stimulation of neuronal activity in a neuronal pathway associated with the reward system. Methods of determining such activation are known in the art including for example neuroimaging or electrophysiology, which can evaluate neuronal activity following exposure to the treatment module compared to prior exposure to treatment.

Activating the reward system may be effected by activation of any component of the reward system which may be for example upstream or downstream to the VTA.

According to specific embodiments, activating the reward system refers to direct activation of dopaminergic neurons of the VTA.

According to other specific embodiments, activating the reward system refers to direct activation of post synaptic neurons of the VTA.

Activation of the reward system may be effected by any method known to one skilled in the art. Non-limiting examples of possible activation methods and agents (collectively referred to as treatment module), which can be used with specific embodiments of the invention include chemical stimulation, neurofeedback, sensory stimulation, auditory stimulation, visual stimulation, magnetic stimulation, electric stimulation, ultrasound, digital experiences and virtual reality stimulation, as further described herein below.

Chemical stimulation may be effected for example by a dopamine agonist capable of crossing the blood brain barrier. Non-limiting examples of dopamine agonists include Cocaine, Heroin, L-dopa, Lisuride, Adamantane, Amino tetralin, Benzazepine, Ergoline, Dihydrexidine derivative, ergot derivative such as Bromocriptine, Ropinirole, Pramipexole, Pergolide, Cabergoline, A-68,930, A-77,636, A-412,997, ABT-670, ABT-724, Aplindore, Apomorphine, Aripiprazole, Bifeprunox, BP-897, CY-208,243, Dizocilpine, Etilevodopa, Flibanserin, Ketamine, Melevodopa, Modafinil, Pardoprunox, Phencyclidine, PD-128,907, PD-168,077, PF-219,061, Piribedil, Pramipexole, Propylnorapomorphine, Pukateine, Quinagolide, Quineloran, Quinpirole, RDS-127, RolO-5824, Ropinirole, Rotigotine, Roxindole, Salvinorin, SKF-89,145, Sumanirole, Terguride, Umespirone, WAY-100,635.

Sensory, auditory and/or visual stimulation may be effected for example by hearing music, feeling non-noxious touch on the skin (e.g. body massage) and different tasks involving gain and loss learning of different visual stimuli.

Digital experiences and/or virtual reality stimulation may be effected as described for example by Beale, I. L. et al. (2007) Journal of Adolescent Health, 41(3), 263-270; Cole, S. W. et al. (2012) PLoS ONE, 7(3); Lorenz, R. C. et al. (2015) Video game training and the reward system. Frontiers in Human Neuroscience, 9(FEB), https://doi(dot)org/10(dot)3389/fnhum(dot)2015(dot)00040.

Neurofeedback may be effected for example by electroencephalography (EEG), functional magnetic resonance imaging (fMRI), functional near-infrared spectrometry, diffusion-weighted magnetic resonance imaging and/or functional magnetic resonance spectrometry by methods known in the art such as disclosed e.g. in International Patent Application Publication No: WO2020178820, the contents of which are fully incorporated herein by reference in their entirety.

Magnetic stimulation may be effected for example by Transcranial magnetic stimulation (TMS). TMS and methods of using TMS are known in the art and disclosed e.g. in U.S. Pat. Nos. 8,771,163; 8,388,510; 8,277,371 and 7,976,451; and International Patent Application Publication Nos: WO 200232504 and WO2015079439, the contents of which are fully incorporated herein by reference in their entirety.

Stimulation may also be effected by transfecting a target neuron with a receptor activated solely by a synthetic ligand (RASSL) and/or designer receptor exclusively activated by designer drugs (DREADD). Such methods are known in the art and are further disclosed in the Examples section which follows and in International Patent Application Publication Nos: WO2015079439, the contents of which are fully incorporated herein by reference in their entirety.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “subject” refers to a mammal, preferably a human being at any age who is diagnosed with the pathology or is at risk of developing the pathology.

As shown in Table 1 hereinbelow and Example 3 of the Examples section which follows, using proteomics analysis the present inventors have found out that VTA activation results in alteration of expression of proteins involved in the pathogenesis of several diseases. Hence, according to specific embodiments, the disease is associated with (or characterized by) aberrant expression or activity of a protein from the list of proteins depicted in Table 1 hereinbelow.

More specifically, according to specific embodiments, inducing an increase in (or increasing) expression or activity of a protein from the “increased” list of protein depicted in Table 1 hereinbelow is beneficial for the treatment of the disease.

According to specific other embodiments, inducing a decrease in (or decreasing) expression or activity of a protein from the “decreased” list of protein depicted in Table 1 hereinbelow is beneficial for the treatment of the disease.

Using Panther Gene Ontology (GO) analysis, the proteins depicted in Table 1 hereinbelow were classified into several biological processes in which they are involved. Hence, according to specific embodiments, the disease is associated with (or characterized by) aberrant biological function from the list of functions depicted in Table 1 hereinbelow.

More specifically, according to specific embodiments, inducing an increase in (or increasing) complement activation, negative feedback on cytokine production and/or regulation of triglyceride biosynthesis, is beneficial for the treatment of the disease. According to other specific embodiments, inducing a decrease in (or decreasing) RNA splicing, endocytosis, catabolism, and/or metabolic processing of nucleotide-sugars, is beneficial for the treatment of the disease.

According to specific embodiments, the disease is cancer.

Examples of cancers that can be treated according to specific embodiments of the invention include sporadic breast cancer, triple-negative breast cancer-luminal-like subtype wild-type p53, hepatocellular carcinoma, larynx cancer papillary, nasopharyngeal carcinoma, cystadenocarcinoma, non-medullary thyroid cancer, myxosarcoma, renal cell carcinoma, nonpapillary renal cell carcinoma, clear cell papillary renal cell carcinoma, skin squamous cell carcinoma, Gliosarcoma, glioblastoma, giant cell glioblastoma, uveal melanoma, chondroblastoma, pancreatic acinar cell adenocarcinoma, petroclival meningioma, granular cell tumor, follicular dendritic cell sarcoma, liver sarcoma, histiocytic sarcoma, epithelioid sarcoma and periapical granuloma, each possibility represents a separate embodiment of the invention.

According to specific embodiments, the disease is not cancer.

According to specific embodiments, the disease is not in co-morbidity with cancer. In other words, the subject is not diagnosed with a cancerous disease.

Examples of non-cancerous diseases that can be treated according to specific embodiments of the invention include liver disease, kidney disease, psoriatic arthritis, systemic lupus erythematosus (SLE), rheumatoid arthritis, anti-neutrophil cytoplasmic antibody-associated vasculitis, synaptic change in multiple sclerosis, amyotrophic lateral sclerosis (ALS), ALS9, ALS1, neonatal Myasthenia Gravis, Crohn's disease, Huntington disease, oculopharyngeal muscular dystrophy, ataxia-spinocerebellar ataxia 7, spinocerebellar ataxia 1, autosomal dominant cerebellar ataxia, hemolytic anemia, septic shock, stroke, Timothy Syndrome, Long Qt Syndrome, schizophrenia, bipolar disorder, major depression, anxiety, autism, ADHD, sepsis, viral lower respiratory tract infections, chronic heart failure, Diabetes type 2, hemodialysis, dilated cardiomyopathy, lipid storage myopathy, peanut allergy, pericarditis, rheumatic disease, Camptodactyly-Arthropathy-Coxa Vara-Pericarditis Syndrome, amyloidosis, Alzheimer's disease, Parkinson's disease, Laryngeal disease, laryngitis, laryngomalacia, spherocytosis, type 5, hereditary spherocytosis, Bardet-Biedl Syndrome, schizophrenia, hyperhomocysteinemia, neural tube defects, homocystinuria, placental abruption, coronary artery disease, xerophthalmia, Noonan syndrome 6, succinic semialdehyde dehydrogenase deficiency, thrombophilia due to thrombin defect, lecithin: cholesterol acyltransferase deficiency, fish-eye disease, Tangier Disease, hypoalphalipoproteinemia, phosphoserine phosphatase deficiency, arthrogryposis, distal arthrogryposis, distal arthrogryposis type 1C, multidrug-resistant tuberculosis, Meester-Locys syndrome, Monckeberg arteriosclerosis, familial Mediterranean fever, mitochondrial complex Ii deficiency, mitochondrial complex Ii deficiency nuclear type 2, amyotrophic neuralgia, brachial plexus neuropathy, thrombosis and nephrotic syndrome type 5, Agenesis of corpus callosum, cardiac, ocular, and genital syndrome (ACOGS), hematuria, myopathy, lactic acidosis, sideroblastic anemia, snail allergy, crustacean allergy, carnitine-acylcarnitine translocase deficiency, Retinitis pigmentosa, Retinitis pigmentosa 24, pulmonary fibrosis, idiopathic and nasopharyngitis chondrodysplasia punctata 2 X-Linked dominant, Mend syndrome, bare lymphocyte syndrome, bare lymphocyte syndrome type Ii, nominal aphasia, gastrointestinal anthrax, barbiturate dependence, intellectual disability syndrome with long QT, myoclonus, myoclonus familial 1, alcoholic pancreatitis, polymicrogyria, perisylvian, with cerebellar hypoplasia and arthrogryposis (NEDSPLB), arthrogryposis, heparin cofactor Ii deficiency, pyridoxamine 5-prime-phosphate oxidase deficiency, acid-labile subunit deficiency, neurodevelopmental disorder with microcephaly hypotonia and variable brain anomalies (NMIHBA), ulnar neuropathy, developmental delay, brain abnormalities including ventriculomegaly and brain atrophy, optic nerve abnormalities, Diamond-Blackfan anemia, Diamond-Blackfan anemia 13, X-linked myopathy with postural muscle atrophy, Emery-Dreifuss muscular dystrophy 5, Smith-Magenis syndrome, spinocerebellar ataxia autosomal recessive 24, developmental and epileptic encephalopathy 44, Charcot-Marie tooth disease axonal type 2P, autosomal dominant mental retardation 20, 3-methylcrotonyl-coa carboxylase deficiency, St. Louis encephalitis, eczema herpeticum, porphyria-acute Hepatic cutanea tarda variegate, tyrosinemia type I, Hermansky-Pudlak syndrome, hyperbiliverdinemia, cholestasis, pneumothorax, mild cognitive impairment, multiple mitochondrial dysfunctions syndrome 2 with hyperglycinemia, familial isolated dilated cardiomyopathy, caspase 8 deficiency, villonodular synovitis, hantavirus pulmonary syndrome, myotonic dystrophy 1 and 2, palmoplantar keratoderma bothnian type, Silver-Russell syndrome 1, xeroderma pigmentosum complementation group E, persistent hyperplastic primary vitreous (PHPV), trichostrongylosis, osteogenesis imperfecta, dentinogenesis imperfecta, osteopetrosis, narcolepsy, otopalatodigital syndrome, progressive myoclonus epilepsy, thyroid Crisis, granulomatous disease, lymphadenitis, skeletal tuberculosis, lymphopenia, nonparalytic poliomyelitis, pulmonary hypertension, Acute febrile neutrophilic dermatosis, glaucomatocyclitic crisis, cone-rod dystrophy 2, fundus dystrophy, esophageal diverticulosis, malnutrition and cachexia, each possibility represents a separate embodiment of the invention.

According to specific embodiments, the disease is a liver disease.

Non-limiting examples of non-cancerous liver diseases that can be treated according to specific embodiments of the invention include hepatitis A, hepatitis B and hepatitis C, alcoholic fatty liver, cirrhosis, steatosis, non-alcoholic steatohepatitis, autoimmune hepatitis, primary biliary cholangitis, primary sclerosing cholangitis, and medication-induced liver injury, each possibility represents a separate embodiment of the invention.

According to specific embodiments, the liver disease is selected from the group consisting of hepatitis A, hepatitis B and hepatitis C.

According to specific embodiments, the disease is a kidney disease.

Non-limiting examples of non-cancerous kidney diseases that can be treated according to specific embodiments of the invention include C3 glomerulopathy, chronic kidney disease, acute glomerulonephritis, membranoproliferative glomerulonephritis, atypical hemolytic uremic syndrome, Lupus nephritis, podocytopathy, diabetic nephropathy, albuminuria, autosomal dominant polycystic kidney disease, ischemia/reperfusion-induced acute kidney injury, chronic renal failure and progressive proteinuric nephropathy, post-Streptococcal glomerulonephritis, atypical hemolytic uremic syndrome, age related macular degeneration, membranoproliferative glomerulonephritis, hereditary angioedema, each possibility represents a separate embodiment of the invention.

According to specific embodiments, the kidney disease is selected from the group consisting of C3 glomerulopathy, chronic kidney disease, acute glomerulonephritis, membranoproliferative glomerulonephritis, atypical hemolytic uremic syndrome, Lupus nephritis, podocytopathy, diabetic nephropathy, albuminuria, autosomal dominant polycystic kidney disease, ischemia/reperfusion-induced acute kidney injury, chronic renal failure and progressive proteinuric nephropathy.

According to specific embodiments, the methods disclosed herein can be combined with other established or experimental therapeutic regimen to treat the diseases disclosed herein including, but not limited to surgical and ablation interventions, analgesics, chemotherapeutic agents, radiotherapeutic agents, cytotoxic therapies (conditioning), hormonal therapy, antibodies, antibiotics, steroids, anti-viral and other treatment regimens which are well known in the art.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

TABLE 1
Full proteomic analysis (LC/MSMS) of cardiac apex samples 4 days post MI indicated
modifications in multiple proteins associated with various diseases

| | | | Student's | | | |
|---|---|---|---|---|---|---|
| | | | T-test | | | |
| | Gene | Protein | p-value LFQ | Fold | | |
| | names | names | control_LFQ Gq | change | Function | Relevant conditions |
| Increased | C3 | Complement | 0.040 | 0.611 | Important | Autoimmune disease- |
| | | C3 | | | component in | psoriatic arthritis, Crohn's |
| | | | | | all complement | disease, systemic lupus |
| | | | | | pathways | erythematosus (SLE), |
| | | | | | | rheumatoid arthritis. |
| | | | | | | Hemolytic anemia. Septic |
| | | | | | | shock. Liver diseases - |
| | | | | | | Hepatitis (A/B/C). |
| | | | | | | Malnutrition. Kidney |
| | | | | | | diseases-C3 |
| | | | | | | glomerulopathy, chronic |
| | | | | | | kidney disease, acute |
| | | | | | | glomerulonephritis, |
| | | | | | | membranoproliferative |
| | | | | | | glomerulonephritis. |
| | | | | | | Synaptic change in |
| | | | | | | multiple sclerosis. Stroke. |
| | Cacna1c | Voltage- | 0.000 | 0.004 | Mediates | Timothy Syndrome, Long |
| | | dependent L- | | | influx of | Qt Syndrome, |
| | | type calcium | | | calcium ions | Schizophrenia. Bipolar |
| | | channel | | | into the | disorder. Major depression. |
| | | subunit alpha- | | | cytoplasm, and | Anxiety. Autism. ADHD. |
| | | 1C | | | thereby | |
| | | | | | triggers | |
| | | | | | calcium release | |
| | | | | | from the | |
| | | | | | sarcoplasm. | |
| | | | | | Plays an | |
| | | | | | important role | |
| | | | | | in excitation- | |
| | | | | | contraction | |
| | | | | | coupling in the | |
| | | | | | heart. Required | |
| | | | | | for normal | |
| | | | | | heart | |
| | | | | | development | |
| | | | | | and normal | |
| | | | | | regulation of | |
| | | | | | heart rhythm. | |
| | Cfp | Properdin | 0.016 | 0.581 | Modulator of | Renal diseases-Atypical |
| | | | | | the complement | hemolytic uremic |
| | | | | | alternative | syndrome, C3 |
| | | | | | pathway | glomerulopathy, Lupus |
| | | | | | | nephritis. Autoimmune- |
| | | | | | | Anti-neutrophil |
| | | | | | | cytoplasmic antibody- |
| | | | | | | associated vasculitis, SLE. |
| | | | | | | Inflammatory disease- |
| | | | | | | sepsis, viral lower |
| | | | | | | respiratory tract infections. |
| | | | | | | Chronic heart failure. |
| | | | | | | Diabetes type 2. |
| | | | | | | Hemodialysis. |
| | Cherp | Calcium | 0.000 | 0.015 | Participate in | Huntington Disease. |
| | | homeostasis | | | cellular | Oculopharyngeal Muscular |
| | | endoplasmic | | | calcium | Dystrophy. Ataxia- |
| | | reticulum | | | homeostasis, | Spinocerebellar Ataxia 7, |
| | | protein | | | mRNA | Spinocerebellar Ataxia 1, |
| | | | | | splicing | Autosomal Dominant |
| | | | | | (spliceosome), | cerebellar Ataxia. Renal |
| | | | | | negative | disease- podocytopathy, |
| | | | | | regulation of | diabetic nephropathy, |
| | | | | | cellular | albuminuria, autosomal |
| | | | | | proliferation, | dominant polycystic |
| | | | | | release of | kidney disease, and |
| | | | | | sequestered | ischemia/reperfusion- |
| | | | | | calcium ion | induced acute kidney |
| | | | | | into cytosol. | injury |
| | | | | | Molecularly | |
| | | | | | binds to ion | |
| | | | | | channels and | |
| | | | | | RNA. | |
| | Emsy | BRCA2- | 0.014 | 1.652 | | Sporadic Breast Cancer |
| | | interacting | | | | and Peanut Allergy |
| | | transcriptional | | | | |
| | | repressor | | | | |
| | | EMSY | | | | |
| | Fhod3 | FH1/FH2 | 0.000 | 0.126 | Actin filament | Dilated cardiomyopathy |
| | | domain- | | | polymerization | |
| | | containing | | | in | |
| | | protein 3 | | | cardiomyocytes | |
| | Flad1 | FAD synthase | 0.027 | 0.579 | Catalyzes the | Lipid Storage Myopathy |
| | | | | | adenylation of | |
| | | | | | flavin | |
| | | | | | mononucleotide | |
| | | | | | (FMN) to | |
| | | | | | form flavin | |
| | | | | | adenine | |
| | | | | | dinucleotide | |
| | | | | | (FAD) | |
| | | | | | coenzyme. | |
| | | | | | Molecular | |
| | | | | | functioning: | |
| | | | | | ATP binding, | |
| | | | | | FMN | |
| | | | | | adenylyltransferase | |
| | | | | | activity | |
| | Prg4 | Proteoglycan 4 | 0.030 | 0.446 | non-structural | Weight gain, hepatocellular |
| | | | | | component of | carcinoma, Pericarditis, |
| | | | | | the | rheumatic diseases and |
| | | | | | extracellular | Camptodactyly- |
| | | | | | matrix, | Arthropathy-Coxa Vara- |
| | | | | | expressed in | Pericarditis Syndrome |
| | | | | | the liver, | |
| | | | | | important for | |
| | | | | | joint | |
| | | | | | homeostasis | |
| | | | | | and is | |
| | | | | | considered | |
| | | | | | cardioprotective. | |
| | | | | | Proteoglycan | |
| | | | | | 4 attenuates | |
| | | | | | pathological | |
| | | | | | cardiac | |
| | | | | | remodeling of | |
| | | | | | myocardial | |
| | | | | | ischemia | |
| | Apcs | Serum | 0.136 | 0.338 | acute phase | amyloidosis, Alzheimer |
| | | amyloid P- | | | protein | |
| | | component | | | activates the | |
| | | | | | complement | |
| | | | | | cascade and | |
| | | | | | enhance | |
| | | | | | phagocytosis | |
| | | | | | by neutrophils. | |
| | | | | | Apcs−/− mice | |
| | | | | | are defective in | |
| | | | | | vivo in terms | |
| | | | | | of recruitment | |
| | | | | | of neutrophils | |
| | | | | | and | |
| | | | | | phagocytosis | |
| | | | | | (34145258). | |
| | | | | | contribute to | |
| | | | | | the clearance | |
| | | | | | of apoptotic | |
| | | | | | cells. Can | |
| | | | | | interact with | |
| | | | | | DNA and | |
| | | | | | histones, | |
| | | | | | Calcium co | |
| | | | | | factor and | |
| | | | | | ligand, acute | |
| | | | | | phase, classical | |
| | | | | | complement | |
| | | | | | activation, | |
| | | | | | negative | |
| | | | | | regulation of | |
| | | | | | acute | |
| | | | | | inflammatory | |
| | | | | | response, viral | |
| | | | | | infection, | |
| | | | | | monocyte | |
| | | | | | differentiation, | |
| | | | | | wound healing | |
| | Apod | Apolipoprotein | 0.054 | 0.355 | HDL lipid | Neurological disorders- |
| | | D | | | transporter | Alzheimer's Disease, |
| | | | | | | Parkinson's disease, stroke, |
| | | | | | | schizophrenia, bipolar |
| | | | | | | disorder |
| | Bhmt; | Betaine-- | 0.123 | 0.109 | Involved in the | Hyperhomocysteinemia. |
| | Bhmt2 | homocysteine | | | regulation of | Neural Tube Defects. |
| | | S- | | | homocysteine | Homocystinuria. Placental |
| | | methyltransferase | | | metabolism | Abruption. |
| | | 1; S- | | | | Vascular disease- coronary |
| | | methylmethionine-- | | | | artery disease |
| | | homocysteine | | | | |
| | | S- | | | | |
| | | methyltransferase | | | | |
| | | BHMT2 | | | | |
| | Bpifa2 | BPI fold- | 0.112 | 0.201 | strong | Xerophthalmia and |
| | | containing | | | antibacterial | Papillary |
| | | family A | | | activity against | Cystadenocarcinoma |
| | | member 2 | | | P. aeruginosa, | |
| | | | | | LPS binding | |
| | Ca3 | Carbonic | 0.124 | 0.134 | Reversible | Laryngeal diseases- larynx |
| | | anhydrase 3 | | | hydration of | cancer, laryngitis, |
| | | | | | carbon dioxide, | laryngomalacia. Acute |
| | | | | | response to | myocardial infarction |
| | | | | | bacterium and | |
| | | | | | oxidative | |
| | | | | | stress, | |
| | | | | | metabolism | |
| | Epb42 | Erythrocyte | 0.107 | 0.130 | role in the | Spherocytosis, Type 5 and |
| | | membrane | | | regulation of | Hereditary Spherocytosis |
| | | protein band | | | erythrocyte | |
| | | 4.2 | | | shape and | |
| | | | | | mechanical | |
| | | | | | properties. | |
| | Fsd2 | Fibronectin | 0.107 | 0.093 | Fibronectin | Bardet-Biedl Syndrome |
| | | type III and | | | type III and | |
| | | SPRY domain- | | | SPRY domain- | |
| | | containing | | | containing | |
| | | protein 2 | | | protein 2. | |
| | | | | | Highly | |
| | | | | | expressed in | |
| | | | | | the heart | |
| | Gbp9 | GB1/RHD3- | 0.130 | 0.186 | Signaling. | Noonan Syndrome 6 |
| | | type G | | | Catalytic | |
| | | domain- | | | activity. | |
| | | containing | | | Response to | |
| | | protein | | | bacterium and | |
| | | | | | IFNγ. | |
| | | | | | Expressed by | |
| | | | | | immune cells | |
| | | | | | but also liver | |
| | | | | | brain etc. | |
| | Gpld1 | Phosphatidylinositol- | 0.097 | 0.401 | Catalytic | Succinic Semialdehyde |
| | | glycan- | | | activity. Cell | Dehydrogenase |
| | | specific | | | migration | Deficiency and Glandular |
| | | phospholipase | | | involved in | Tularemia |
| | | D | | | sprouting | |
| | | | | | angiogenesis, | |
| | | | | | hematopoietic | |
| | | | | | stem cell | |
| | | | | | migration, | |
| | | | | | cellular | |
| | | | | | response to | |
| | | | | | cholesterol and | |
| | | | | | calcium. | |
| | | | | | Expressed in | |
| | | | | | the liver and | |
| | | | | | cardiovascular | |
| | | | | | and more | |
| | Habp2 | Hyaluronan- | 0.084 | 0.396 | expressed in | Thyroid Cancer, |
| | | binding | | | the liver. | Nonmedullary, 5 and |
| | | protein 2 | | | Activates | Thrombophilia Due To |
| | | | | | coagulation | Thrombin Defect |
| | | | | | factor, not | |
| | | | | | initiate fibrin | |
| | | | | | clot, cell | |
| | | | | | adhesion, | |
| | | | | | proteolysis | |
| | Lcat | Phosphatidylcholine- | 0.070 | 0.069 | Highly in the | Lecithin: Cholesterol |
| | | sterol | | | liver. A key | Acyltransferase |
| | | acyltransferase | | | enzyme in | Deficiency. Fish-Eye |
| | | | | | esterification | Disease. Tangier Disease. |
| | | | | | of free | Hypoalphalipoproteinemia. |
| | | | | | cholesterol, | Renal disease-chronic renal |
| | | | | | mostly HDL | failure, progressive |
| | | | | | | proteinuric nephropathy. |
| | Mrps17 | 28S ribosomal | 0.116 | 0.115 | rRNA binding, | Myxosarcoma and |
| | | protein S17, | | | structural, | Phosphoserine Phosphatase |
| | | mitochondrial | | | constituent of | Deficiency |
| | | | | | ribosome, | |
| | | | | | mitochondrial | |
| | | | | | translation | |
| | Mylpf | Myosin | 0.063 | 0.195 | structural | Arthrogryposis, Distal, |
| | | regulatory | | | constituent of | Type 1C and Distal |
| | | light chain 2, | | | muscle, | Arthrogryposis |
| | | skeletal | | | calcium ion | |
| | | muscle | | | binding, | |
| | | isoform | | | muscle | |
| | | | | | contraction, | |
| | | | | | actin | |
| | | | | | nucleation | |
| | Pbrm1 | Protein | 0.092 | 0.049 | chromatin and | Renal Cell Carcinoma, |
| | | polybromo-1 | | | DNA binding, | Nonpapillary and Clear |
| | | | | | negative | Cell Papillary Renal Cell |
| | | | | | regulation of | Carcinoma |
| | | | | | cell | |
| | | | | | proliferation, | |
| | | | | | stimulates | |
| | | | | | transcription of | |
| | | | | | androgen receptor | |
| | Pglyrp2 | N- | 0.077 | 0.138 | Expressed in | Multidrug-Resistant |
| | | acetylmuramoyl- | | | the liver. | Tuberculosis and Meester- |
| | | L-alanine | | | Peptidoglycan | Loeys Syndrome |
| | | amidase | | | bonding, innate | |
| | | | | | immune | |
| | | | | | response, | |
| | | | | | antimicrobial | |
| | | | | | humoral | |
| | | | | | response, | |
| | | | | | negative | |
| | | | | | regulation of | |
| | | | | | IFNγ | |
| | | | | | production and | |
| | | | | | NK | |
| | | | | | differentiation | |
| | Rarres2 | Retinoic acid | 0.107 | 0.154 | Secreted also | Monckeberg |
| | | receptor | | | by the liver. | Arteriosclerosis and Skin |
| | | responder | | | Elevates | Squamous Cell Carcinoma |
| | | protein 2 | | | platelet | |
| | | | | | cytosolic Ca2+ | |
| | | | | | Adipocyte- | |
| | | | | | secreted | |
| | | | | | protein that | |
| | | | | | regulates | |
| | | | | | adipogenesis, | |
| | | | | | metabolism | |
| | | | | | and dual role in | |
| | | | | | inflammation | |
| | | | | | (pro-increase | |
| | | | | | secretion of | |
| | | | | | cytokines and | |
| | | | | | anti by and | |
| | | | | | chemotactic | |
| | | | | | factor | |
| | | | | | inhibition of | |
| | | | | | NFkB) | |
| | Rnase4 | Ribonuclease | 0.124 | 0.492 | High in liver | Amyotrophic Lateral |
| | | 4 | | | cancer, | Sclerosis 9 and |
| | | | | | favorable. | Amyotrophic Lateral |
| | | | | | Expressed in | Sclerosis 1 |
| | | | | | the liver etc. | |
| | | | | | Rnase, mRNA | |
| | | | | | cleavage | |
| | Saa4 | Serum | 0.107 | 0.041 | Major acute | Familial Mediterranean |
| | | amyloid A-4 | | | phase reactant, | Fever and Amyloidosis |
| | | protein | | | chemoattractant | |
| | | | | | activity | |
| | Sdhaf1 | Succinate | 0.113 | 0.113 | mitochondrial | Mitochondrial Complex Ii |
| | | dehydrogenase | | | respiratory | Deficiency, Nuclear Type |
| | | assembly | | | chain complex | 2 and Mitochondrial |
| | | factor 1, | | | II assembly | Complex Ii Deficiency |
| | | mitochondrial | | | | |
| | Septin4 | Septin-4 | 0.099 | 0.171 | Filament- | Amyotrophic Neuralgia |
| | | | | | forming | and Brachial Plexus |
| | | | | | cytoskeletal | Neuropathy |
| | | | | | GTPase, | |
| | | | | | required for the | |
| | | | | | induction of | |
| | | | | | cell death | |
| | | | | | mediated by | |
| | | | | | TGFb and by | |
| | | | | | other apoptotic | |
| | | | | | stimuli. | |
| | Serpina10 | Protein Z- | 0.117 | 0.333 | Expressed in | Thrombosis and Nephrotic |
| | | dependent | | | the liver. | Syndrome, Type 5, With or |
| | | protease | | | Favorable in | Without Ocular |
| | | inhibitor | | | cancer. blood | Abnormalities |
| | | | | | coagulation, | |
| | | | | | cellular protein | |
| | | | | | metabolic | |
| | | | | | process, | |
| | | | | | negative | |
| | | | | | regulation of | |
| | | | | | endopeptidase | |
| | | | | | activity, post- | |
| | | | | | translational | |
| | | | | | protein | |
| | | | | | modification | |
| | Tacc1 | Transforming | 0.086 | 0.051 | Receptor | Gliosarcoma and Giant |
| | | acidic coiled- | | | binding: | Cell Glioblastoma |
| | | coil-containing | | | estrogen, | |
| | | protein 1 | | | glucorticoid, | |
| | | | | | nuclear, | |
| | | | | | retinoic acid, | |
| | | | | | thyroid | |
| | | | | | hormone. Cell | |
| | | | | | division and | |
| | | | | | proliferation, | |
| | | | | | microtubule | |
| | | | | | cytoskeleton | |
| | | | | | organization | |
| | Taok1 | Serine/threonine- | 0.074 | 0.381 | Serine/threonine- | Agenesis Of Corpus |
| | | protein | | | protein | Callosum, Cardiac, Ocular, |
| | | kinase TAO1 | | | kinase | And Genital Syndrome |
| | | | | | involved in | |
| | | | | | various | |
| | | | | | processes, | |
| | | | | | DNA damage, | |
| | | | | | apoptosis, | |
| | | | | | neurons | |
| | | | | | cellular | |
| | | | | | homeostasis, | |
| | | | | | regulation of | |
| | | | | | actin and | |
| | | | | | microtubule | |
| | | | | | cytoskeleton | |
| | | | | | organization | |
| | Tmem14c | Transmembrane | 0.118 | 0.095 | heme | Hematuria, Benign |
| | | protein 14C | | | biosynthetic | Familial and Melanoma, |
| | | | | | process, | Uveal |
| | | | | | erythrocyte | |
| | | | | | differentiation | |
| | Yars2 | Tyrosine-- | 0.133 | 0.257 | Binding: ATP, | Myopathy, lactic acidosis, |
| | | RNA ligase, | | | RNA, tyrosine. | and sideroblastic anemia |
| | | mitochondrial | | | Mitochondrial | |
| | | | | | tyrosyl-tRNA | |
| | | | | | aminoacylation, | |
| | | | | | translation | |
| Decreased | Cops7a | COP9 | 0.017 | 3.943 | Component of | Snail and Crustacean |
| | | signalosome | | | the COP9 | Allergy |
| | | complex | | | signalosome | |
| | | subunit 7a | | | complex a | |
| | | | | | complex | |
| | | | | | involved in | |
| | | | | | various cellular | |
| | | | | | and | |
| | | | | | developmental | |
| | | | | | processes. The | |
| | | | | | CSN complex | |
| | | | | | is an essential | |
| | | | | | regulator of the | |
| | | | | | ubiquitin | |
| | | | | | conjugation | |
| | | | | | pathway. DNA | |
| | | | | | damage | |
| | | | | | recognition, | |
| | | | | | post- | |
| | | | | | translational | |
| | | | | | protein | |
| | | | | | modification, | |
| | | | | | viral process. | |
| | Cpsf6 | Cleavage and | 0.016 | 3.759 | mRNA | Carnitine-Acylcarnitine |
| | | polyadenylation | | | processing | Translocase Deficiency |
| | | specificity | | | | and Retinitis Pigmentosa |
| | | factor subunit | | | | 24 |
| | | 6 | | | | |
| | Dpp9 | Dipeptidyl | 0.001 | 15.410 | peptidase, | Pulmonary Fibrosis, |
| | | peptidase 9 | | | highly | Idiopathic and |
| | | | | | expressed in | Nasopharyngitis |
| | | | | | the heart | |
| | Ebp | 3-beta- | 0.000 | 28.582 | Cholesterol | Chondrodysplasia Punctata |
| | | hydroxysteroid- | | | biosynthesis | 2, X-Linked Dominant and |
| | | Delta(8), Delta(7)- | | | and | Mend Syndrome |
| | | isomerase | | | metabolism | |
| | H2ac21 | Histone H2A | 0.000 | 19.594 | Protein | Bare Lymphocyte |
| | | type 2-B | | | metabolism, | Syndrome, Type Ii |
| | | | | | nucleosome, | |
| | | | | | chromatin | |
| | | | | | silencing, | |
| | | | | | limiting DNA | |
| | | | | | accessibility- | |
| | | | | | transcription | |
| | | | | | regulation | |
| | H3- | Histone | 0.030 | 2.052 | nucleosome, | Chondroblastoma |
| | 3b; H3- | H3.3; Histone | | | limiting DNA | |
| | 5 | H3.3C | | | accessibility- | |
| | | | | | transcription | |
| | | | | | regulation, | |
| | | | | | positive | |
| | | | | | regulation of | |
| | | | | | cell growth | |
| | Ilvbl | 2- | 0.000 | 31.366 | Fatty acid | Nominal Aphasia and |
| | | hydroxyacyl- | | | alpha | Gastrointestinal Anthrax |
| | | CoA lyase 2 | | | oxydation.mg+ | |
| | | | | | co factor. | |
| | | | | | Highly | |
| | | | | | expressed in | |
| | | | | | the heart | |
| | Inpp1 | Inositol | 0.047 | 2.505 | Catabolic, | Barbiturate Dependence |
| | | polyphosphate | | | involved | and Bipolar Disorder |
| | | 1-phosphatase | | | phosphatidylinositol, | |
| | | | | | mg+ cofactor, | |
| | | | | | inositol | |
| | | | | | phosphate | |
| | Naa10; | N-alpha- | 0.000 | 16.181 | Catalytic of | Intellectual disability |
| | Naa11 | acetyltransferase | | | acyltransferase | syndrome with Long QT |
| | | 10; N-alpha- | | | which activity | |
| | | acetyltransferase | | | is important for | |
| | | 11 | | | development | |
| | Nol3 | Nucleolar | 0.044 | 1.774 | Binding: | Myoclonus, Familial, 1 and |
| | | protein 3 | | | calcium, | Myoclonus |
| | | | | | caspase, death | |
| | | | | | effector | |
| | | | | | domain and | |
| | | | | | receptor, RNA. | |
| | | | | | RNA, splicing, | |
| | | | | | blood vessels | |
| | | | | | remodeling, | |
| | | | | | negative | |
| | | | | | regulation of | |
| | | | | | cardiac muscle | |
| | | | | | cell apoptosis, | |
| | | | | | apoptosis, | |
| | | | | | hypoxia- | |
| | | | | | induced | |
| | | | | | apoptic | |
| | | | | | signaling, | |
| | | | | | muscle | |
| | | | | | atrophy. | |
| | | | | | Response to | |
| | | | | | ischemia, | |
| | | | | | hypoxia | |
| | | | | | regulation of | |
| | | | | | muscle | |
| | | | | | adaptation. | |
| | | | | | Highly | |
| | | | | | expressed in | |
| | | | | | the heart, | |
| | | | | | decreased in | |
| | | | | | heart failure. | |
| | Nqo2 | Ribosyldihydro- | 0.048 | 23.273 | Enzyme in | Alcoholic Pancreatitis. |
| | | nicotinamide | | | quinone | Breast cancer-luminal-like |
| | | dehydrogenase | | | reductase- | subtype, triple-negative, |
| | | [quinone] | | | catabolic. | wild-type p53 |
| | | | | | Biosynthesis of | |
| | | | | | vitamin K. | |
| | | | | | Chlorine and | |
| | | | | | zinc binding. | |
| | | | | | Positive | |
| | | | | | regulation of | |
| | | | | | vascular | |
| | | | | | associated | |
| | | | | | smooth muscle | |
| | | | | | cell | |
| | | | | | proliferation. | |
| | | | | | Metabolism | |
| | Pi4ka | Phosphatidylinositol | 0.0362578465194376+E53: | 2.114 | Kinase, first | Polymicrogyria, |
| | | 4-kinase | G58E53: | | step in inositol | Perisylvian, With |
| | | alpha | K70E53: | | production | Cerebellar Hypoplasia and |
| | | | K82E53: | | signal | Arthrogryposis and |
| | | | K94C46E53: | | transduction. | Heparin Cofactor Ii |
| | | | E55E53: | | binding:ATP, | Deficiency |
| | | | K106C46E53: | | cadherin. Viral | |
| | | | E55E53: | | replication | |
| | | | K118C4E53: | | | |
| | | | E55 | | | |
| | Pnpo | Pyridoxine-5- | 0.000 | 13.774 | Metabolism, | Pyridoxamine 5-Prime- |
| | | phosphate | | | Catalyzes | Phosphate Oxidase |
| | | oxidase | | | oxidation of | Deficiency and Acid- |
| | | | | | pyrisoxal | Labile Subunit Deficiency |
| | Prune1 | Exopoly- | 0.033 | 1.814 | Phosphatase | Neurodevelopmental |
| | | phosphatase | | | activity, | Disorder With |
| | | PRUNE1 | | | cofactor mg+, | Microcephaly, Hypotonia, |
| | | | | | regulation of | And Variable Brain |
| | | | | | microtubule | Anomalies and Ulnar |
| | | | | | polymerization | Neuropathy |
| | | | | | and | |
| | | | | | neurogenesis | |
| | Ptpn23 | Tyrosine- | 0.005 | 6.647 | cellular | Developmental delay, |
| | | protein | | | response to | intellectual disability, brain |
| | | phosphatase | | | cytokines, | abnormalities including |
| | | non-receptor | | | protein | ventriculomegaly and brain |
| | | type 23 | | | transport, | atrophy, optic nerve |
| | | | | | negative | abnormalities |
| | | | | | regulation of | |
| | | | | | epithelial cell | |
| | | | | | migration. | |
| | | | | | Positive | |
| | | | | | regulation of | |
| | | | | | adherence | |
| | | | | | junctions | |
| | | | | | organization, | |
| | | | | | endosome, Wnt | |
| | | | | | secretion. | |
| | Rps29 | 40S ribosomal | 0.037 | 1.980 | Structure of | Diamond-Blackfan Anemia |
| | | protein S29 | | | ribosime, binds | 13 and Diamond-Blackfan |
| | | | | | zinc. | Anemia |
| | | | | | Translation! | |
| | Sdf2l1 | Stromal cell- | 0.000 | 37.608 | Chaperone | Nasopharyngeal |
| | | derived factor | | | binding, ER | Carcinoma |
| | | 2-like protein | | | associated | |
| | | 1 | | | catabolism | |
| | | | | | regulation of | |
| | | | | | apoptosis. | |
| | Tmem214 | Transmembrane | 0.046 | 9.620 | ER stress | Myopathy, X-Linked with |
| | | protein 214 | | | induced | Postural Muscle |
| | | | | | apoptosis | Atrophy and Emery- |
| | | | | | | Dreifuss Muscular |
| | | | | | | Dystrophy 5 |
| | Tom1l2 | TOM1-like | 0.048 | 4.294 | Protein | Smith-Magenis |
| | | protein 2 | | | transport, | Syndrome and Pancreatic |
| | | | | | signaling, | Acinar Cell |
| | | | | | negative | Adenocarcinoma |
| | | | | | regulation of | |
| | | | | | mitotic nuclear | |
| | | | | | division | |
| | Uba5 | Ubiquitin-like | 0.005 | 26.417 | Catalyze | Spinocerebellar Ataxia, |
| | | modifier- | | | ufmylaion, | Autosomal Recessive 24 |
| | | activating | | | Binding ATP | and Developmental and |
| | | enzyme 5 | | | and zinc. | Epileptic Encephalopathy |
| | | | | | Differentiation: | 44 |
| | | | | | erythrocyte and | |
| | | | | | megakaryocyte, | |
| | | | | | ER stress | |
| | | | | | response, | |
| | | | | | neuromuscular | |
| | | | | | process | |
| | Ube2k | Ubiquitin- | 0.007 | 37.957 | Protein | Charcot-Marie-Tooth |
| | | conjugating | | | ubiquitination. | Disease, Axonal, Type 2P |
| | | enzyme E2 K | | | Bind ATP. | and Autosomal Dominant |
| | | | | | Apoptotic | Mental Retardation 20 |
| | | | | | signaling to ER | |
| | | | | | stress, positive | |
| | | | | | regulation of | |
| | | | | | TNF pathway, | |
| | | | | | IFN type 1. | |
| | | | | | cellular | |
| | | | | | response to | |
| | | | | | IFN-B | |
| | Ubxn1 | UBX domain- | 0.034 | 3.636 | negative | 3-Methylcrotonyl-Coa |
| | | containing | | | regulation of | Carboxylase Deficiency |
| | | protein 1 | | | ubiquitination. | and St. Louis Encephalitis |
| | | | | | Bind ATP. | |
| | | | | | Double-strand | |
| | | | | | break repair, | |
| | | | | | protein folding, | |
| | | | | | viral process, | |
| | | | | | metabolism | |
| | Adprhl1 | ADP- | 0.071 | 2.001 | binds mg+ | Posterior Myocardial |
| | | ribosylarginine] | | | | Infarction |
| | | hydrolase- | | | | |
| | | like protein 1 | | | | |
| | Adss2 | Adenylosuccinate | 0.116 | 10.642 | | Eczema Herpeticum |
| | | synthetase | | | | |
| | | isozyme 2 | | | | |
| | Alad | Delta- | 0.083 | 13.455 | Catalyzes | Porphyria- Acute Hepatic, |
| | | aminolevulinic | | | tetrapyrroles, | Cutanea Tarda, Variegate. |
| | | acid | | | binds zinc. | Tyrosinemia, Type I |
| | | dehydratase | | | Heme | |
| | | | | | biosynthesis, | |
| | | | | | response to: | |
| | | | | | IL-4, metal | |
| | | | | | ions, AA, | |
| | | | | | drugs, | |
| | | | | | glucocorticoids, | |
| | | | | | hypoxia, FA, | |
| | | | | | ER stress, | |
| | | | | | vitamins | |
| | Ap3s1 | AP-3 complex | 0.119 | 6.705 | Golgi | Hermansky-Pudlak |
| | | subunit sigma- | | | associated, | Syndrome |
| | | 1 | | | trafficking to | |
| | | | | | lysosomes, | |
| | | | | | anterograde | |
| | | | | | exonal | |
| | | | | | transport, | |
| | | | | | insulin | |
| | | | | | signaling, | |
| | | | | | protein and | |
| | | | | | vesicle | |
| | | | | | transport | |
| | Blvra | Biliverdin | 0.135 | 8.772 | Protoheme | Hyperbiliverdinemia. |
| | | reductase A | | | degradation | Cholestasis. |
| | | | | | (heme | Pneumothorax. Petroclival |
| | | | | | catabolism), | Meningioma. Mild |
| | | | | | NAD | Cognitive Impairment |
| | | | | | reductase, | |
| | | | | | binds zinc | |
| | Bola2 | BolA-like | 0.098 | 8.886 | iron-sulfur | Multiple Mitochondrial |
| | | protein 2 | | | cluster | Dysfunctions Syndrome 2 |
| | | | | | binding-protein | With Hyperglycinemia |
| | | | | | maturation, IL- | |
| | | | | | 12 signaling, | |
| | | | | | cellular iron | |
| | | | | | homeostasis | |
| | Cap2 | Adenylyl | 0.059 | 2.176 | cAMP | Familial Isolated Dilated |
| | | cyclase- | | | signaling, actin | Cardiomyopathy |
| | | associated | | | and adenylate | |
| | | protein 2 | | | cyclase | |
| | | | | | binding, cell | |
| | | | | | morphogenesis, | |
| | | | | | cytoskeleton | |
| | | | | | organization | |
| | Casp8 | Caspase-8 | 0.118 | 11.420 | programed cell | Alzheimer's Disease. |
| | | | | | death, | Caspase 8 Deficiency. |
| | | | | | apoptosis also | Hepatocellular Carcinoma |
| | | | | | extrinsic, | |
| | | | | | angiogenesis, | |
| | | | | | B, T cells, NK | |
| | | | | | and | |
| | | | | | macrophages | |
| | | | | | activation, | |
| | | | | | proteolysis, | |
| | | | | | regulation of | |
| | | | | | cytokine | |
| | | | | | production, | |
| | Cd68 | Macrosialin | 0.128 | 21.087 | Phagocytic | Granular Cell Tumor. |
| | | | | | activity if | Follicular Dendritic Cell |
| | | | | | macrophages. | Sarcoma. Liver Sarcoma. |
| | | | | | Cellular | Histiocytic Sarcoma. |
| | | | | | response to | Villonodular Synovitis |
| | | | | | LPS, nutrient | |
| | | | | | levels. Aging, | |
| | | | | | inflammatory | |
| | | | | | response to | |
| | | | | | antigenic | |
| | | | | | stimulus, | |
| | | | | | negative | |
| | | | | | regulation of | |
| | | | | | DCs antigen | |
| | | | | | processing and | |
| | | | | | presentation, | |
| | | | | | neutrophils | |
| | | | | | degranulation | |
| | Cdh5 | Cadherin-5 | 0.120 | 6.992 | calcium- | Epithelioid Sarcoma. |
| | | | | | dependent cell | Periapical Granuloma. |
| | | | | | adhesion | Acute Myocardial |
| | | | | | proteins, | Infarction. Hantavirus |
| | | | | | angiogenesis | Pulmonary Syndrome |
| | | | | | blood vessels | |
| | | | | | endothelial cell | |
| | | | | | migration and | |
| | | | | | maturation, | |
| | | | | | cell-cell | |
| | | | | | adhesion. | |
| | | | | | Negative | |
| | | | | | regulation of | |
| | | | | | proliferation, | |
| | | | | | endothelial cell | |
| | | | | | apoptosis, | |
| | | | | | inflammatory | |
| | | | | | response, | |
| | | | | | microtucle | |
| | | | | | polymerization. | |
| | Celf1 | CUGBP Elav- | 0.095 | 6.926 | Pre-mRNA | Myotonic Dystrophy 1 and |
| | | like family | | | alternative | 2 |
| | | member 1 | | | splicing. | |
| | | | | | Regulation of | |
| | | | | | gene | |
| | | | | | expression and | |
| | | | | | cell death and | |
| | | | | | inflammatory. | |
| | | | | | Activates | |
| | | | | | TNNT2 during | |
| | | | | | heart | |
| | | | | | remodeling | |
| | | | | | response. | |
| | Chtop | Chromatin | 0.120 | 10.518 | mRNA nuclear | Glioblastoma |
| | | target of | | | export. Play | |
| | | PRMT1 | | | role in ligand- | |
| | | protein | | | depended | |
| | | | | | estrogen | |
| | | | | | receptor target | |
| | | | | | genes. Positive | |
| | | | | | regulation of | |
| | | | | | ATPase and | |
| | | | | | cell | |
| | | | | | proliferation | |
| | Copg2 | Coatomer | 0.118 | 7.267 | ER to Golgi | Palmoplantar Keratoderma, |
| | | subunit | | | transport, | Bothnian Type. Silver- |
| | | gamma-2 | | | protein | Russell Syndrome 1 |
| | | | | | secretion | |
| | Cops2 | COP9 | 0.105 | 2.607 | Transcription | Xeroderma Pigmentosum, |
| | | signalosome | | | corepressor | Complementation Group E. |
| | | complex | | | activity. | Persistent Hyperplastic |
| | | subunit 2 | | | component of | Primary Vitreous |
| | | | | | the COP9 | |
| | | | | | signalosome | |
| | | | | | complex a | |
| | | | | | complex | |
| | | | | | involved in | |
| | | | | | ubiquitin | |
| | | | | | process | |
| | Coro6 | Coronin-6 | 0.120 | 3.482 | Actin filament | Neonatal Myasthenia |
| | | | | | binding- | Gravis |
| | | | | | organization, | |
| | | | | | cell migration | |
| | Cpped1 | Serine/threonine- | 0.116 | 10.772 | Protein | Trichostrongylosis |
| | | protein | | | phosphatase | |
| | | phosphatase | | | that | |
| | | CPPED1 | | | dephosphorylates | |
| | | | | | AKT family | |
| | | | | | kinase | |
| | | | | | blocking cell | |
| | | | | | cycle | |
| | | | | | progression | |
| | | | | | and promoting | |
| | | | | | cell apoptosis. | |
| | | | | | Metal binding. | |
| | | | | | Neutrophil | |
| | | | | | degranulation | |
| | Crtap | Cartilage- | 0.123 | 13.769 | Necessary for | Osteogenesis Imperfecta. |
| | | associated | | | hydroxylation | Dentinogenesis Imperfecta |
| | | protein | | | of fibrillar | |
| | | | | | collagen. | |
| | | | | | Protein folding | |
| | | | | | (chaperone) | |
| | | | | | protein | |
| | | | | | stabilization | |
| | | | | | and negative | |
| | | | | | regulation of | |
| | | | | | post | |
| | | | | | translational | |
| | | | | | modification | |
| | Csk | Tyrosine- | 0.136 | 11.461 | Non-receptor | Osteopetrosis. SLE |
| | | protein kinase | | | binds ATP and | |
| | | CSK | | | Mg+. Negative | |
| | | | | | regulation of | |
| | | | | | cell growth, | |
| | | | | | differentiation, | |
| | | | | | migration and | |
| | | | | | immune response | |
| | | | | | (mainly B and | |
| | | | | | T) | |
| | Ctsh | Pro-cathepsin | 0.118 | 11.783 | Degradation of | Narcolepsy. |
| | | H | | | proteins in | Otopalatodigital |
| | | | | | lysosomes. | Syndrome. Progressive |
| | | | | | Immune- | Myoclonus Epilepsy. |
| | | | | | adaptive, | Thyroid Crisis |
| | | | | | antigen | |
| | | | | | presentation, T | |
| | | | | | cell | |
| | | | | | cytotoxicity, | |
| | | | | | neutrophil | |
| | | | | | degranulation. | |
| | | | | | Negative | |
| | | | | | regulation of | |
| | | | | | apoptosis. | |
| | | | | | Positive | |
| | | | | | regulation: | |
| | | | | | angiogenesis, | |
| | | | | | cell migration, | |
| | | | | | gene | |
| | | | | | expression and | |
| | | | | | peptidase | |
| | | | | | activity. | |
| | | | | | Response to | |
| | | | | | bradykinin, | |
| | | | | | thyroid | |
| | | | | | hormone, | |
| | | | | | retinoic acid. | |
| | Cybc1 | Cytochrome b- | 0.117 | 8.807 | Stable | Granulomatous Disease. |
| | | 245 chaperone | | | expression of | Lymphadenitis |
| | | 1 | | | cytochrome. | |
| | | | | | Essential got | |
| | | | | | innate | |
| | | | | | immunity and | |
| | | | | | phagocytes | |
| | | | | | respiratory | |
| | | | | | burst | |
| | Cygb | Cytoglobin | 0.052 | 21.743 | Protective | Retinitis Pigmentosa. |
| | | | | | during | Cone-Rod Dystrophy 2. |
| | | | | | oxidative | Fundus Dystrophy. |
| | | | | | stress. Heme, | Esophageal Diverticulosis |
| | | | | | iron and | |
| | | | | | oxygen | |
| | | | | | binding. | |
| | | | | | Peroxide | |
| | | | | | activity. | |
| | | | | | Oxygen | |
| | | | | | transport and | |
| | | | | | regulation of | |
| | | | | | NO synthase | |
| | | | | | activity. | |
| | | | | | Expressed in | |
| | | | | | cardiac muscle | |
| | Dock2 | Dedicator of | 0.132 | 9.224 | Cytoskeletal | HIV associated |
| | | cytokinesis | | | rearrangements | immunodeficiency. |
| | | protein 2 | | | required for | Skeletal Tuberculosis. |
| | | | | | lymphocyte | Lymphopenia |
| | | | | | migration in | |
| | | | | | response of | |
| | | | | | chemokines. T | |
| | | | | | cell receptor | |
| | | | | | binding leading | |
| | | | | | to activation | |
| | | | | | and | |
| | | | | | proliferation. | |
| | | | | | Positive | |
| | | | | | regulation of | |
| | | | | | phagocytosis | |
| | Dynlt1 | Dynein light | 0.119 | 14.185 | neuronal | Nonparalytic Poliomyelitis. |
| | | chain Tctex- | | | morphogenesis. | Pulmonary Hypertension. |
| | | type 1 | | | Negative | Neutrophilic Dermatosis, |
| | | | | | regulation of | Acute Febrile. |
| | | | | | cell death, | Glaucomatocyclitic Crisis |
| | | | | | neurogenesis. | |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Mice—Adult (10-12 weeks of age; 20-25 g) male and female DAT-Cre mice (strain B6.SJL-Slc6a3tm1.1(cre)Bkmn/J, The Jackson Laboratory) were used in all experiments. Mice were housed in a Specific-Pathogen-Free (SPF) conditions on a 12:12 hours light:dark cycle with a room temperature of 22±1° C. Mice received standard mouse chow and water added libitum. All experiments were performed in accordance with the National Institutes of Health Guide for the Care and Use of Laboratory Animals. All procedures and protocols were approved by the Technion

Administrative Panel of Laboratory Animal Care. All efforts were made to minimize suffering. Mice were acclimatized for 1-2 weeks before experiments.

Stereotactic injection—Mice were randomly divided between VTA-activated and control group. Mice were anesthetized using a ketamine/xylazine mixture (ketamine 80 mg/kg; xylazine 15-20 mg/kg) diluted in sterile saline solution (NaCl 0.9%), given analgesic with buprenorphine (0.05 mg/kg subcutaneous) before being fixed in a stereotactic frame (Stoelting, Wood Dale, IL, USA). In addition, a Duratears eye ointment was used to prevent dehydration and the incision site was sterilized. An AAV8 virus (AAV8-hSyn-DIO-hM3D(Gq)-mCherry, the Vector Core at the University of North Carolina, https://www(dot)vvf(dot)uzh(dot)ch/en(dot)html #GQ V89-8) was used for Cre-dependent DREADD expression. 0.4 μl of the virus (1012-1013 vg/ml) was injected into the right VTA region (anterior-posterior—3.2 mm; medial-lateral 0.48 mm; dorsal-ventral 4.65 mm) of DAT-Cre mice. Control mice were injected with a sham AAV8-hSyn-DIO-mCherry construct, lacking the DREADD gene ((https://www(dot)vvf(dot)uzh(dot)ch/en(dot)html #CONTROL V116-8). This allowed control of local inflammatory response, induced by the virus and CNO injection. Experiments were performed 30 days following virus injection to ensure the expression of the DREADD. Mice showing signs of physical distress and pain were excluded from the experiment. Following sacrifice, stereotactic injection sites were verified by immunohistochemistry. In all experiments, control mice were subjected to the same CNO regimen.

Myocardial infarction—The left anterior descending (LAD) coronary artery permanent ligation was used as a model for acute myocardial infarction (MI). Prior to the procedure, mice were heavily sedated in an isoflurane chamber followed by endotracheal intubation and artificial ventilation with oxygen 1-100% and Isoflurane 1-2.5% throughout the procedure. The mice were placed on a heated surface 37° C., given analgesic with buprenorphine (0.05 mg/kg subcutaneous) and eye ointment. The chest was shaved and sterilized by iodine solution, a small thorax incision was made, and the skin and muscle layers were gently separated. Left thoracotomy was made in the third intercostal space (3rd and 4th ribs) and gently spread apart using a retractor. The pericardium was removed with forceps. The left coronary artery was identified and permanently ligated with a monofilament nylon 8-0. The rib cage was closed by 6-0 nylon suture and skin with a tissue adhesive. At the end of the procedure 200-500 μL of 0.9% heated saline was administrated. When the mouse demonstrated an adequate bilateral depth breathing rate and responded to a toe pinch, the intubation tube was removed, and the mouse was placed in a clean recovery cage under a heat lamp. Following, mice were administrated with buprenorphine 0.05 mg/mL subcutaneously for three days. Following the procedure mice were daily assessed for weight, respiration and behavior.

MRI—Functional cardiac magnetic resonance (fCMR) was performed on days 1 and 15 following LAD ligation to assess LVEF. MRI short-axis views of the entire left ventricle were acquired on a Bruker 9.4 Tesla PhamaScan magnet (Billerica) with a cine fast low-angle shot sequence with intraGate technology using the following parameters: echo time: 2.945 ms; repetition time: 10.0 ms; flip angle: 18 degrees; oversampling: 250; frames: 16; matrix: 200×200×1; and voxel size: 0.150×0.150×1 mm. The left ventricular wall and cavity were segmented manually at systole and diastole using Segment-CMR by Medviso software (https://medviso(dot)com/cmr/).

Tissue preparation and immunohistochemistry—Validation of the virus injection site by evaluation of DREADDs expression and TH/c-Fos analysis; and assessment of blood vascularization in the heart using vWF and α-SMA were performed by immunohistochemistry. Mice were sacrificed, and their brains were fixed in 4% paraformaldehyde (PFA) in PBS for 24 hours, cryoprotected in 30% sucrose solution for another 48 hours, and then frozen in dry ice. Coronal cryosections from the midbrain were sliced at 12 μm thickness and mounted on super-frost slides (Fisherbrand). The tissues were stained for TH with mouse anti-TH (1:200; Millipore, Billerica, MA, USA) and the proportion of DREADD-expressing cells (mCherry+) out of the total TH+ population in the VTA was evaluated (min 1500 cells). To evaluate c-Fos expression, the mice were sacrificed 90 minutes following CNO injection, and treated as described above. The fixed sections were prepared and stained with rabbit anti-c-Fos antibodies (1:100, Calbiochem,San Diego, CA, USA). The proportion of c-Fos+ cell nuclei was calculated from the total number of virus-expressing cells. Quantification of double positive cells was performed using Fiji software. Hearts were fresh frizzed in OCT with liquid nitrogen and kept at −80° C. Cryosections from the heart were sliced at 10 μm thickness and mounted on super-frost slides (Fisherbrand). The tissue was fixed with 50% methanol and 50% acetone for 10 minutes at −20° C. The tissues were stained for vWF, α-SMA, CD68, CD45 with anti-mouse antibodies and secondary by the source. All images were taken at ×20 or ×10 magnification using an Axio imager M2 microscope (Carl Zeiss Inc. US).

Scar tissue calculation—The difference in scar-tissue between VTA-activated and control mice was calculated as follows: the total myocardial area and the collagen-rich area in histologic preparations obtained from the hearts of mice, were estimated following the research protocol. Based on the dominant presence of collagen in scar-tissue, Masson trichrome staining was used to demonstrate the scar-tissue within the myocardium. Following, for each group the proportion of collagen-rich area relative to the total myocardial area was calculated as follows:

$$\% collagen - rich area = \frac{collagen - rich {area}_{{μ m}^{2}}}{total myocardial {area}_{{μ m}^{2}}} \times 100$$

The area was calculated using Weka plug-in of FIJI (ImageJ) software, in which a classifier was generated based on machine learning, distinguishing between blue collagen, red myocardium and white background. The RGB output was used as a threshold to quantify the area bigger than 100 ROI.

Proteomics—The samples were trypsinized and analyzed by the LC/MSMS using the QE HFX (Thermo) mass spectrometer. The data was analyzed using the MaxQuant and the Perseus software.

PRV injections—Mice were anesthetized with a ketamine-xylazine mixture (ketamine 80 mg per kg body weight (mg/kg); xylazine 15-20 mg/kg; Sigma-Aldrich) in sterile saline (0.9% NaCl). Injections of Pseudorabies virus Bartha strain 152 (PRV-152 (GFP) to the liver hilum was performed by adaptation of the procedure previously described by Muller et al. (2020). Following shaving and sterilization of the abdomen, mice were placed on a sterile surgical pad on top of a heating pad. Ophthalmic ointment (Duratears, Alcon) was placed over the eyes to prevent dehydration and the incision site was sterilized. Upon loss of recoil paw compression, a midline incision was made bellow the diaphragm, exposing the peritoneal cavity. For injection to the liver hilum, the liver hilum was located, exposed for injection and held in place using two stabilized swabs. An overall of 1 mL of virus was injected using a syringe (Hamilton) and a Stoelting Quintessential Stereotaxic Injector (QSI), into two different locations along the hilum. Following injection, the abdominal wall and the skin were separately closed using absorbable sutures. Antibiotic ointment was applied to the closed surgical site and mice were given 0.05 mg/kg buprenorphine every 12 hours for three days. Liver, ganglion, spinal cord and brains were collected 6 days following injection.

### Example 1

### Neuronal Connection Between the VTA and the Liver

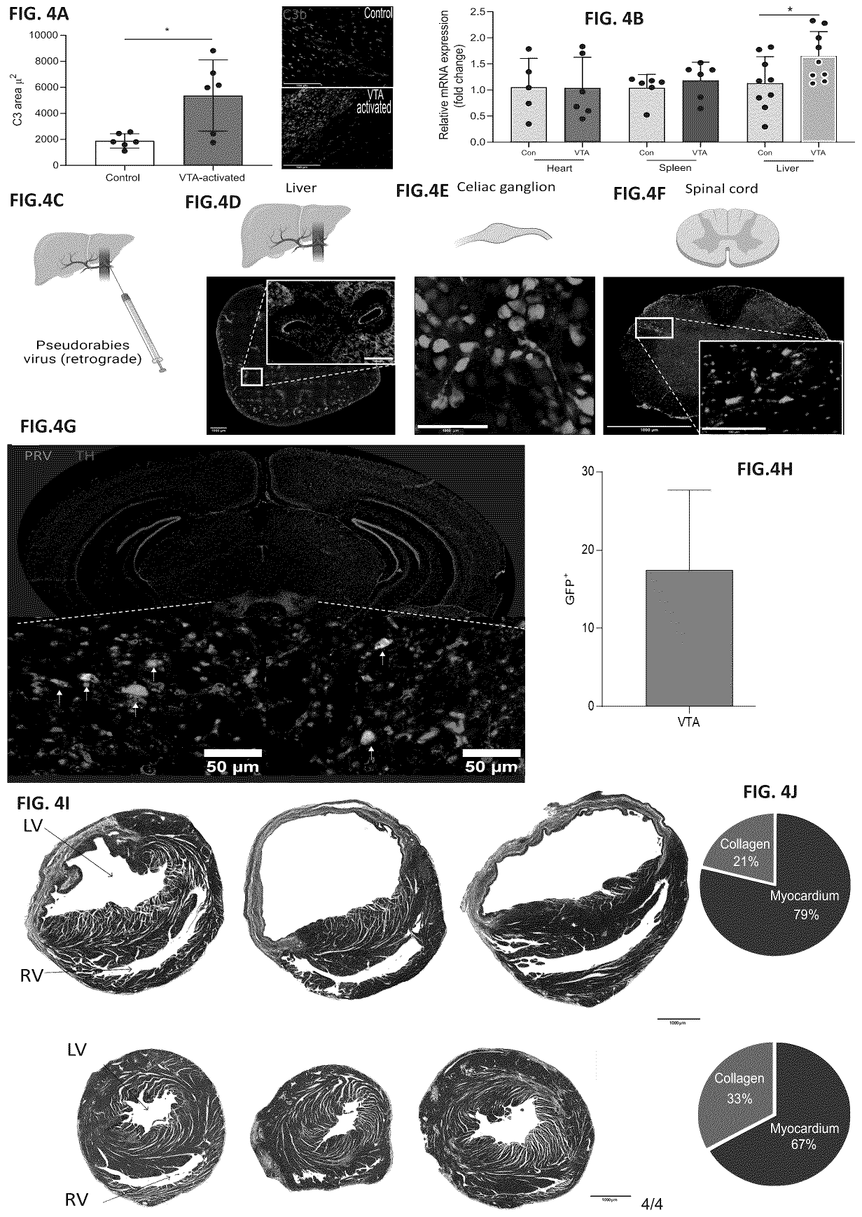

The liver produces proteins associated with better recovery following acute myocardial infarction (MI). To determine whether a connection exists between the liver and emotional processing networks, a Pseudorabies virus Bartha strain 152 (PRV152 (GFP)) expressing a GFP reporter was injected to hepatic nerves located at the liver's hilum. PRV shows a retrograde spread and is therefore expected to reach the brain regions that transduce its neuronal output. The main focus was on brain regions involved in emotional processing due to the correlation between the emotional state and outcomes following acute MI. Neurons in the PVN and the amygdala were observed, two brain regions that are closely associated with stress and were previously described [la Fleur, S. E. et al. (2000) Brain Research (Vol. 871) www(dot)elsevier(dot)com/locate/bres; Buijs, R. M. et al. (2003) Journal of Comparative Neurology, 464(1), 36-48]. Yet, cells in the VTA were also identified, a brain region mediating positively-valenced emotions, which has been linked to immune system function (FIGS. 4A-J).

### Example 2

### VTA Activation as Treatment for Myocardial Infarction

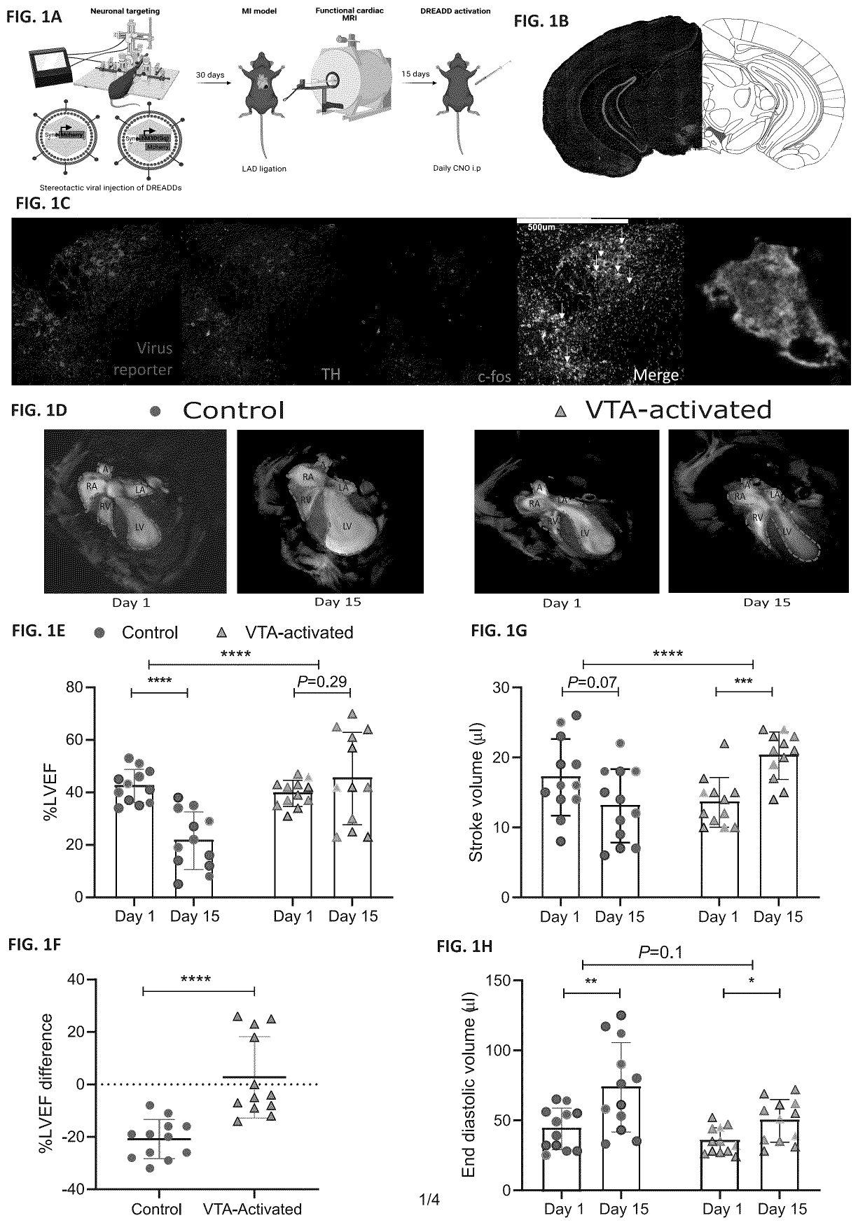

To determine the effect of VTA activation on cardiac recovery following acute myocardial infarction (MI), a chemogenetic technique termed ‘Designer Receptors Exclusively Activated by Designer Drugs’ (DREADD) was used to directly activate dopaminergic neurons in the VTA in a mouse AMI model (FIG. 1A). Briefly, a viral vector comprising an excitatory G-protein coupled receptor (Gq) with mCherry reporter information was injected stereotacticly into the VTA of DAT mice expressing cre under the promotor of dopamine transporter (DAT). This allowed exclusive targeting of dopaminergic neurons in the VTA followed by activation using the receptor's artificial ligand clozapine-N-oxide (CNO). Control mice were injected with a virus encoding the mCherry reporter without DREADD's information. Viral expression was validated by the mCherry reporter and neuronal activation was validated using immunofluorescence by c-Fos, an early activation marker, and tyrosine hydroxylase (TH), the rate limiting enzyme in dopamine production to validate DREADD activation and specificity (FIGS. 1B-C). Following recovery from the procedure and upon viral expression, mice were operated to effect permanent left anterior descending artery (LAD) ligation. Following LAD ligation, the DREADD receptor in VTA dopaminergic neurons was daily activated for a total of 15 days.

As LAD is a commonly affected vessel supplying the anterior wall of the left ventricle, LAD ligation leads to ischemia to the left ventricle bearing a significant damage to cardiac function, which is most evident in the reduction in left-ventricular ejection fraction (LVEF), as seen for example in the control group (FIGS. 1D-E). The LVEF represents the fraction of blood volume pumped out of the left ventricle and into the aorta, entering the bloodstream; and thus serves as a key parameter in the evaluation of left-ventricular (LV) dysfunction. To this end, functional cardiac magnetic resonance imaging (fCMR), a gold-standard method for left-ventricular function evaluation, was effected on day 1 post MI to assess the initial damage of LAD ligation model and 14 days following MI to study the left-ventricular functional outcome following treatment (FIG. 1D-H). Both male and female mice with initial LVEF (day 1) lower than 55% were included in the study. A significant LVEF difference was observed between control and VTA activated mice [2-way ANOVA mixed model p=****<0.0001 Cl 95% 2.153 to 13.01] (FIGS. 1D-E). The control mice exhibited a reduction in LVEF on day 15 [student's t-test p=****<0.0001]. This result is in line with other studies indicating further LVEF reduction following permanent LAD ligation. On the contrary, VTA-activated mice exhibited no significant change in LVEF on day 15 [student's t-test p=0.29], meaning no associated deterioration and even an LVEF improvement in some cases (FIGS. 1E and 1F). Moreover, VTA-activated mice showed a significant difference in stroke volume (SV) compared to the control group [2-way ANOVA mixed model p=****<0.0001], (FIG. 1G). VTA-activated mice had a higher SV on day 14 than on day 1 [student's t-test p=***] meaning more volume of blood was pumped out of the left ventricle during each systolic cardiac contraction. There was no significant difference in the end-diastolic-volume (EDV) between the groups (FIG. 1H) [2-way ANOVA mixed model p=0.1]. Taken together, the data indicates a beneficial healing process in the VTA activated group.

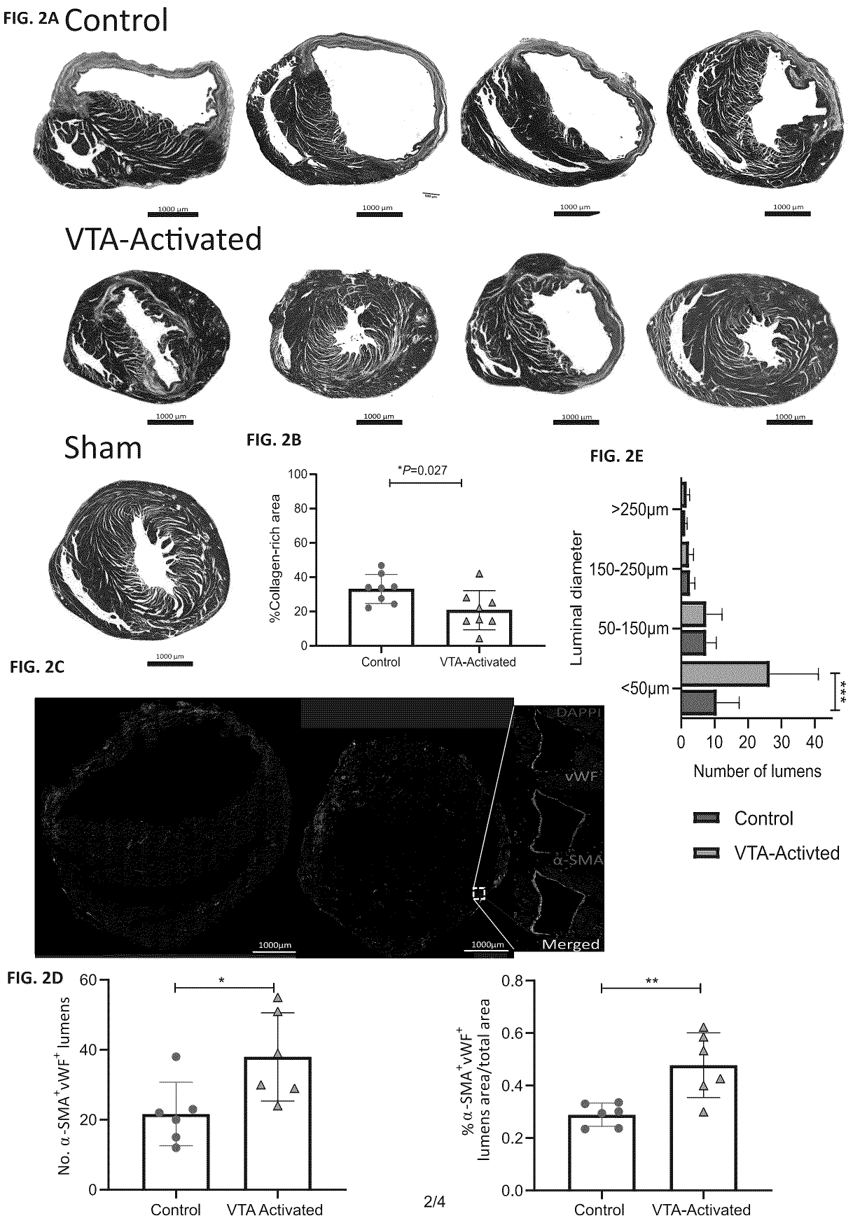

To further assess whether VTA activation effects the scar pattern. Masson's trichrome histological staining was effected on heart samples obtained on day 15 following LAD ligation. Control mice showed continuous scars occupying most of the left ventricle, in line with the literature. In contrast, hearts obtained from VTA-activated mice showed scattered patterns of scaring with more functional myocardium area (FIG. 2A). Quantifying the scar area and the myocardial area using image analysis indicated that indeed VTA-activated mice had a smaller scar area and a bigger functional myocardium area (FIG. 2B). To study the vascularization that supports cardiac recovery, cardiac histologic samples obtained 15 days post MI were stained for von-Willebrand factor (vWF) and α-smooth muscle actin (α-SMA) using immunofluorescence (FIG. 2C). As endothelial cells form the vascular lumen and produce vesicles expressing vWF; and expression of α-SMA is mainly by vascular smooth muscle cells and to a lesser degree by myofibroblasts, only clear lumen morphology was quantified and used for analysis. Cardiac tissue samples of VTA-activated mice showed a significantly larger area stained positive for vWF and a-SMA, indicating greater vascularity (FIG. 2D). Moreover, VTA-activated mice showed a greater number of vascular lumens compared to control mice (FIG. 2D). Looking at the distribution in diameter of these vascular lumens, a greater fraction of small size (<50 um) lumens was observed in samples from VTA-activated mice, suggesting the formation of new vessels was more dominant (FIG. 2E). These findings may provide at least a partial explanation for the differences in the cardiac functional outcomes observed between VTA-activated and control mice.

### Example 3

VTA Activation Mofidies Expression of Proteins Associated with Various Diseases

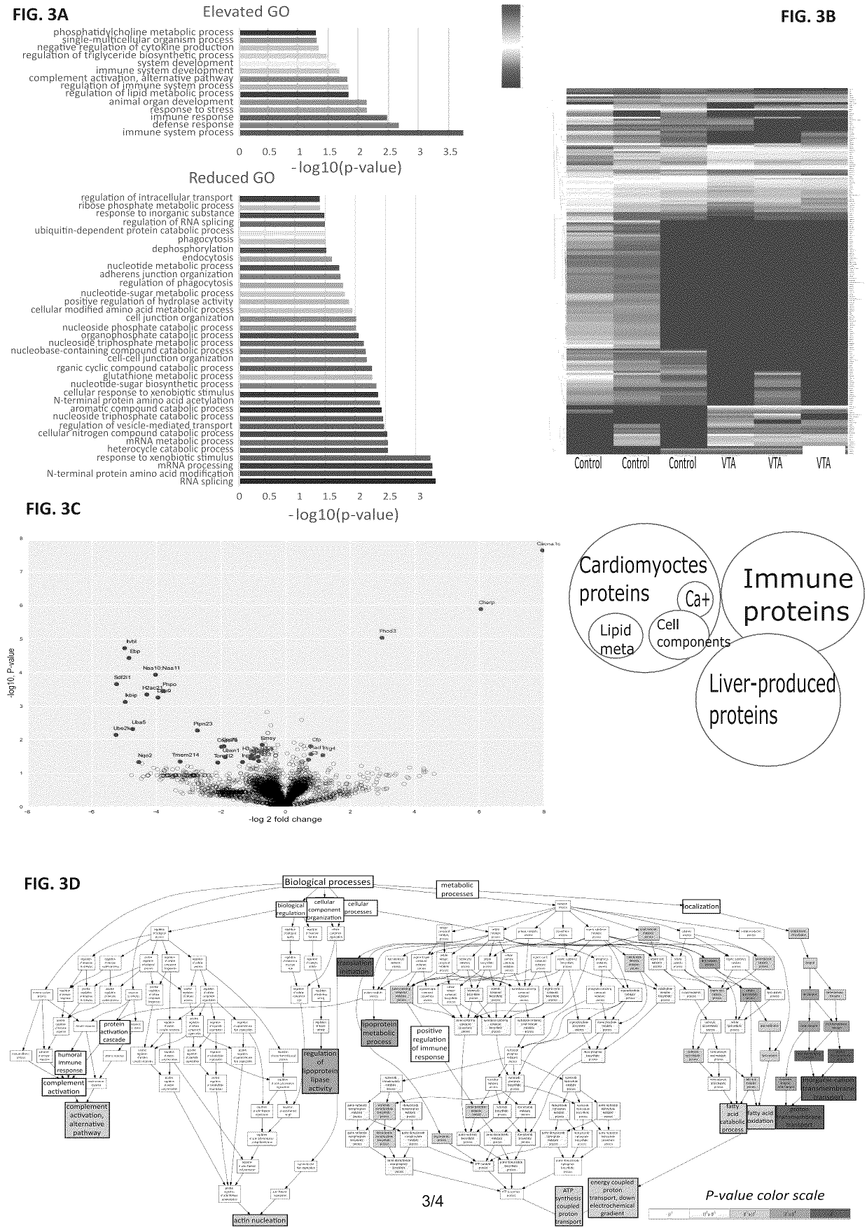

To further investigate the processes that mediate the beneficial effects of VTA activation following MI, a time point of 4 days post-MI was selected, which serves as the interphase between the inflammatory and proliferative phases of MI and is considered as a key step in the process of wound healing. Thus, targeting factors that modify this phase transition may offer a rationale for the development of therapeutic interventions. Full proteomic analysis (LC/MSMS) of cardiac apex samples effected at this time point showed modifications in multiple proteins (see Table 1 hereinabove and FIG. 3A). Using Panther Gene Ontology (GO) analysis, these proteins were classified into several biological processes in which they are involved. Protein levels that differed significantly between VTA-activated and control mice were mostly associated with cellular and metabolic types of processes. These proteins were also involved in intracellular signal transduction, regulatory processes, immune system function, structure formation, etc. (FIG. 3B). Specifically, GO annotation in VTA-activated mice points to an increase in the amount of immune system processes, including complement activation, negative feedback on cytokine production, organ development, and regulation of triglyceride biosynthesis. On the other hand, VTA-activated mice showed a decrease in number of processes, including RNA splicing, endocytosis, catabolism, and metabolic processing of nucleotide-sugars. Pathway enrichments analysis indicated that VTA activation results in alterations in many processes that are related to the recovery process following MI (FIG. 3C). For example, biological processes resulting in complement activation through the alternative pathway and actin nucleation, the first step in actin polymerization, as well as synthesis of lipoproteins and ATP.

Taken together, these results suggest that VTA activation effects an array of pathways intimately involved in the recovery process following MI. Moreover, the proteomics analysis indicated that VTA activation results in alteration of expression of proteins involved in several other diseases-indicating VTA activation as a therapeutic strategy for these diseases.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating a disease selected from the group consisting of liver disease, kidney disease, psoriatic arthritis, systemic lupus erythematosus (SLE), rheumatoid arthritis, anti-neutrophil cytoplasmic antibody-associated vasculitis, synaptic change in multiple sclerosis, amyotrophic lateral sclerosis (ALS), ALS9, ALS1, neonatal Myasthenia Gravis, Crohn's disease, Huntington disease, oculopharyngeal muscular dystrophy, ataxia-spinocerebellar ataxia 7, spinocerebellar ataxia 1, autosomal dominant cerebellar ataxia, hemolytic anemia, septic shock, stroke, Timothy Syndrome, Long Qt Syndrome, schizophrenia, bipolar disorder, major depression, anxiety, autism, ADHD, sepsis, viral lower respiratory tract infections, chronic heart failure, Diabetes type 2, hemodialysis, dilated cardiomyopathy, lipid storage myopathy, peanut allergy, pericarditis, rheumatic disease, Camptodactyly-Arthropathy-Coxa Vara-Pericarditis Syndrome, amyloidosis, Alzheimer's disease, Parkinson's disease, Laryngeal disease, laryngitis, laryngomalacia, spherocytosis, type 5, hereditary spherocytosis, Bardet-Biedl Syndrome, schizophrenia, hyperhomocysteinemia, neural tube defects, homocystinuria, placental abruption, coronary artery disease, xerophthalmia, Noonan syndrome 6, succinic semialdehyde dehydrogenase deficiency, thrombophilia due to thrombin defect, lecithin:cholesterol acyltransferase deficiency, fish-eye disease, Tangier Disease, hypoalphalipoproteinemia, phosphoserine phosphatase deficiency, arthrogryposis, distal arthrogryposis, distal arthrogryposis type 1C, multidrug-resistant tuberculosis, Meester-Locys syndrome, Monckeberg arteriosclerosis, familial Mediterranean fever, mitochondrial complex Ii deficiency, mitochondrial complex Ii deficiency nuclear type 2, amyotrophic neuralgia, brachial plexus neuropathy, thrombosis and nephrotic syndrome type 5, Agenesis of corpus callosum, cardiac, ocular, and genital syndrome (ACOGS), hematuria, myopathy, lactic acidosis, sideroblastic anemia, snail allergy, crustacean allergy, carnitine-acylcarnitine translocase deficiency, Retinitis pigmentosa, Retinitis pigmentosa 24, pulmonary fibrosis, idiopathic and nasopharyngitis chondrodysplasia punctata 2 X-Linked dominant, Mend syndrome, bare lymphocyte syndrome, bare lymphocyte syndrome type Ii, nominal aphasia, gastrointestinal anthrax, barbiturate dependence, intellectual disability syndrome with long QT, myoclonus, myoclonus familial 1, alcoholic pancreatitis, polymicrogyria, perisylvian, with cerebellar hypoplasia and arthrogryposis (NEDSPLB), arthrogryposis, heparin cofactor Ii deficiency, pyridoxamine 5-prime-phosphate oxidase deficiency, acid-labile subunit deficiency, neurodevelopmental disorder with microcephaly hypotonia and variable brain anomalies (NMIHBA), ulnar neuropathy, developmental delay, brain abnormalities including ventriculomegaly and brain atrophy, optic nerve abnormalities, Diamond-Blackfan anemia, Diamond-Blackfan anemia 13, X-linked myopathy with postural muscle atrophy, Emery-Dreifuss muscular dystrophy 5, Smith-Magenis syndrome, spinocerebellar ataxia autosomal recessive 24, developmental and epileptic encephalopathy 44, Charcot-Marie tooth disease axonal type 2P, autosomal dominant mental retardation 20, 3-methylcrotonyl-coa carboxylase deficiency, St. Louis encephalitis, eczema herpeticum, porphyria-acute Hepatic cutanea tarda variegate, tyrosinemia type I, Hermansky-Pudlak syndrome, hyperbiliverdinemia, cholestasis, pneumothorax, mild cognitive impairment, multiple mitochondrial dysfunctions syndrome 2 with hyperglycinemia, familial isolated dilated cardiomyopathy, caspase 8 deficiency, villonodular synovitis, hantavirus pulmonary syndrome, myotonic dystrophy 1 and 2, palmoplantar keratoderma bothnian type, Silver-Russell syndrome 1, xeroderma pigmentosum complementation group E, persistent hyperplastic primary vitreous (PHPV), trichostrongylosis, osteogenesis imperfecta, dentinogenesis imperfecta, osteopetrosis, narcolepsy, otopalatodigital syndrome, progressive myoclonus epilepsy, thyroid Crisis, granulomatous disease, lymphadenitis, skeletal tuberculosis, lymphopenia, nonparalytic poliomyelitis, pulmonary hypertension, Acute febrile neutrophilic dermatosis, glaucomatocyclitic crisis, cone-rod dystrophy 2, fundus dystrophy, esophageal diverticulosis, malnutrition and cachexia, wherein said disease is not cancer, in a subject in need thereof, the method comprising subjecting the subject to a treatment module which activates the reward system of the subject, thereby treating the disease in the subject.

2. The method of claim 1,
wherein said liver disease is selected from the group consisting of hepatitis A, hepatitis B and hepatitis C; and/or
wherein said kidney disease is selected from the group consisting of C3 glomerulopathy, chronic kidney disease, acute glomerulonephritis, membranoproliferative glomerulonephritis, atypical hemolytic uremic syndrome, Lupus nephritis, podocytopathy, diabetic nephropathy, albuminuria, autosomal dominant polycystic kidney disease, ischemia/reperfusion-induced acute kidney injury, chronic renal failure and progressive proteinuric nephropathy.

3. The method of claim 1, wherein said treatment module activates a dopaminergic neuron in the ventral tegmental area (VTA) of said subject or a post synaptic neuron thereof.

4. The method of claim 1, wherein said treatment module comprises sensory, auditory, visual and/or chemical stimulation.

5. The method of claim 1, wherein said treatment module comprises digital experiences and/or virtual reality stimulation.

6. The method of claim 1, wherein said treatment module comprises magnetic stimulation, electric stimulation and/or an ultrasound stimulation.

7. The method of claim 1, wherein said treatment module comprises transfecting a neuron in said reward system with a receptor activated solely by a synthetic ligand (RASSL) and/or designer receptor exclusively activated by designer drugs (DREADD).

8. The method of claim 1, wherein said treatment module comprises neurofeedback.

9. The method of claim 8, wherein said neurofeedback comprises electroencephalography, functional magnetic resonance imaging, functional near-infrared spectrometry, diffusion-weighted magnetic resonance imaging and/or functional magnetic resonance spectrometry.

10. The method of claim 1, wherein said treatment module comprises administering to the subject a dopamine agonist capable of crossing the blood brain barrier.

11. A method of treating cancer in a subject in need thereof, wherein said cancer is selected from the group consisting of sporadic breast cancer, triple-negative breast cancer-luminal-like subtype wild-type p53, hepatocellular carcinoma, larynx cancer papillary, nasopharyngeal carcinoma, cystadenocarcinoma, non-medullary thyroid cancer, myxosarcoma, renal cell carcinoma, nonpapillary renal cell carcinoma, clear cell papillary renal cell carcinoma, skin squamous cell carcinoma, Gliosarcoma, glioblastoma, giant cell glioblastoma, uveal melanoma, chondroblastoma, pancreatic acinar cell adenocarcinoma, petroclival meningioma, granular cell tumor, follicular dendritic cell sarcoma, liver sarcoma, histiocytic sarcoma, epithelioid sarcoma and periapical granuloma, the method comprising subjecting the subject to a treatment module which activates the reward system of the subject, thereby treating the cancer in the subject.

12. The method of claim 11, wherein said treatment module activates a dopaminergic neuron in the ventral tegmental area (VTA) of said subject or a post synaptic neuron thereof.

13. The method of claim 11, wherein said treatment module comprises sensory, auditory, visual and/or chemical stimulation.

14. The method of claim 11, wherein said treatment module comprises digital experiences and/or virtual reality stimulation.

15. The method of claim 11, wherein said treatment module comprises magnetic stimulation, electric stimulation and/or an ultrasound stimulation.

16. The method of claim 11, wherein said treatment module comprises transfecting a neuron in said reward system with a receptor activated solely by a synthetic ligand (RASSL) and/or designer receptor exclusively activated by designer drugs (DREADD).

17. The method of claim 11, wherein said treatment module comprises neurofeedback.

18. The method of claim 17, wherein said neurofeedback comprises electroencephalography, functional magnetic resonance imaging, functional near-infrared spectrometry, diffusion-weighted magnetic resonance imaging and/or functional magnetic resonance spectrometry.

19. The method of claim 11, wherein said treatment module comprises administering to the subject a dopamine agonist capable of crossing the blood brain barrier.
